(19) United States
(12) Patent Application Publication
(10) Pub. No.: US 2024/0366543 A1
(43) Pub. Date: Nov. 7, 2024

(54) METHODS OF TREATING METHYLMALONIC ACIDEMIA AND PROPIONIC ACIDEMIA

(71) Applicant: HemoShear Therapeutics, Inc., Charlottesville, VA (US)

(72) Inventors: Allison ARMSTRONG, Charlottesville, VA (US); Brian JOHNS, Charlottesville, VA (US); Gerald COX, Charlottesville, VA (US)

(21) Appl. No.: 18/285,807

(22) PCT Filed: Apr. 6, 2022
(86) PCT No.: PCT/US2022/023703

§ 371 Date: Oct. 5, 2023

Related U.S. Application Data
(60) Provisional application No. 63/171,326, filed on Apr. 6, 2021.
(60) Provisional application No. 63/255,153, filed on Oct. 13, 2021.
(60) Provisional application No. 63/288,978, filed on Dec. 13, 2021.

(51) Int. Cl. A61K 31/19; A61P 3/00

(52) U.S. Cl. CPC A61K 31/19; A61P 3/00

(57) ABSTRACT

The present disclosure relates to methods of treating organic acidemias with 2.2-dimethylbutyric acid or pharmaceutically acceptable salt thereof.

### CROSS-REFERENCE TO RELATED APPLICATIONS

The present Application claims priority to U.S. Provisional Patent Application No. 63/171,326, filed Apr. 6, 2021, U.S. Provisional Patent Application No. 63/255,153 filed Oct. 13, 2021, and U.S. Provisional Patent Application No. 63/288,978 filed Dec. 13, 2021, the entire contents of which are incorporated herein by reference and relied upon.

### BACKGROUND

Metabolic disorders, such as organic acidemias, occur when there is a mutation in an enzyme that causes a significant loss of function which interrupts the normal flux of metabolites in a metabolic pathway. This results in accumulation of normal intermediary metabolites in abnormally large amounts and in some cases, the production of abnormal metabolites that are not normally formed when there is not a mutation that causes a significant loss of function in an enzyme.

For example, propionic acidemia (PA) and methylmalonic acidemia (MMA) are inborn errors of metabolism that result in the buildup of metabolites. The incidence rates for PA are 1 in 242,741 individuals in the US, 1 in 50,000 to 100,000 people worldwide, and the incidence can be as high as 1 in 1,000 to 2,000 in specific populations that are genetically at higher risk (e.g., Inuit population of Greenland, some Amish communities, Saudi Arabians, and communities with consanguineous marriage), whereas MMA affects 1 in 69,354 births.

PA is caused by a dysfunction of the propionyl-CoA carboxylase (EC 6.4.1.3) enzyme which blocks the conversion of propionyl-CoA to methylmalonyl-CoA resulting in the accumulation of propionyl-CoA in cells and metabolites such as 3-hydroxypropionic acid, 2-methylcitric acid, and propionylcarnitine in the urine and in the blood. Inhibition of the urea cycle (assumed to be by 3-hydroxypropionic acid or propionyl-CoA) results in clinically significant elevations in blood ammonia, contributing to both morbidity and mortality.

MMA is caused by dysfunction of the vitamin B12-dependent methylmalonyl-CoA mutase (EC 5.4.99.2) enzyme, which blocks the conversion of methylmalonyl-CoA to succinyl-CoA resulting in the accumulation of metabolites such as propionyl-CoA, methylmalonyl-CoA, methylmalonic acid, 3-hydroxypropionic acid, 2-methylcitric acid, and propionylcarnitine in the blood and tissues. A complete or partial enzyme deficiency results in the mut⁰ or mut⁻ disease subtype, respectively. In some instances, MMA can be caused by a dysfunction of the methylmalonyl-CoA epimerase (EC 5.1.99.1) enzyme, also called methylmalonyl racemase. In addition, MMA can also be caused by defective synthesis of adenosylcobalamin (an active form of vitamin B12) by MMAA, MMAB and MMADHC. Similar to PA, the accumulation of certain toxic metabolites in MMA patients results in reduced urea cycle function (assumed to be by 3-hydroxypropionic acid or propionyl-CoA), which can cause clinically significant elevations in blood ammonia, contributing to both morbidity and mortality.

Patients suffering from PA or MMA have elevated levels of certain metabolites resulting from defective enzymes (propionyl-CoA carboxylase or methylmalonyl-CoA mutase, respectively). Patients with PA and MMA often present acutely with metabolic acidosis, dehydration, lethargy, seizures, vomiting, and hyperammonemia causing severe central nervous system dysfunction. Long term complications include seizures, cardiomyopathies, metabolic stroke like episodes, cardiac arrhythmias, chronic kidney failure, impaired consciousness, ketosis, pancreatitis, and optic atrophy, which severely impact the quality of life and cause progressive deterioration, sometimes ending in sudden death.

There are no current definitive therapies for PA or MMA. Mostly, treatment options focus on severe dietary and lifestyle modifications and symptomatic management of the complications and sequelae arising due to acute and long-term exposure to toxic metabolites associated with the disease state. The dietary regimen involves restricting the precursors of propionyl-CoA, such as branched-chain amino acids (valine and isoleucine), threonine, methionine, odd-chain fatty acids and cholesterol, while trying to maintain normal growth. Dietary supplementation with levocarnitine, biotin (PA) and/or cobalamin (MMA) is also common. In addition, propiogenic gut bacteria is controlled with antibiotic regimens, and complications are treated symptomatically as they occur. Despite the symptomatic relief, many of these patients still progress to the long-term sequelae of the disease.

Liver and/or kidney transplantation may be required. For example, some patients with PA receive orthotopic liver transplantation (OLT) to ameliorate symptoms primarily due to hyperammonemia.

Therefore, developing an effective therapeutic method to treat PA and MMA is critical for improving clinical manifestations of the disease as well as improving the quality of life and life span of these patients. Thus, there exists a need to treat metabolic disorders (e.g., PA and MMA) by reducing the levels of toxic metabolites associated with the disease state. The present disclosure solves this need.

### SUMMARY OF DISCLOSURE

In some embodiments, the present disclosure provides methods of treating an organic acidemia (e.g., propionic acidemia (PA), isovaleric acidemia (IVA), or methylmalonic acidemia (MMA), or any other disease disclosed herein) in a subject in need thereof, comprising administering to a subject an effective amount of sodium 2,2-dimethylbutanoate, or an equivalent dose of a different pharmaceutically acceptable salt thereof, 2,2-dimethylbutyric acid, or a CoA ester or carnitine ester thereof.

### BRIEF DESCRIPTIONS OF THE FIGURES

FIG. 1 shows the effects of Compound 5 (sodium 2,2-dimethylbutanoate) on the concentrations of propionyl-CoA from various sources in primary hepatocytes of propionic acidemia patients. All primary hepatocytes were treated with Compound 5 ranging from 0 μM to 1,000 μM. FIG. 1A shows the concentration of propionyl-CoA in primary hepatocytes in the presence of 1 mM ¹³C-KIVA. The concentration of propionyl-CoA had an EC₅₀ of 0.89 μM. FIG. 1B shows the concentration of propionyl-CoA in primary hepatocytes in the presence of 3 mM ¹³C-ILE. The concentration of propionyl-CoA had an EC₅₀ of 0.42 μM. FIG. 1C shows the concentration of propionyl-CoA in primary hepatocytes in the presence of 5 mM ¹³C-THR. The concentration of propionyl-CoA had an EC₅₀ of 1.24 μM. FIG. 1D shows the concentration of propionyl-CoA in primary hepatocytes in the presence of 5 mM ¹³C-propionate. The concentration of propionyl-CoA had an EC₅₀ of 15.27 μM.

FIG. 2 shows the effects of Compound 5 on the concentrations of propionyl-CoA and methylmalonyl-CoA from various sources in primary hepatocytes of methylmalonic acidemia patients. All primary hepatocytes were treated with Compound 5 ranging from 0 μM to 1,000 μM. FIG. 2A shows the concentrations of propionyl-CoA and methylmalonyl-CoA in primary hepatocytes in the presence of 1 mM ¹³C-KIVA. The concentration of propionyl-CoA had an EC₅₀ of 0.93 μM. The concentration of and methylmalonyl-CoA had an EC₅₀ of 1.17 μM. FIG. 2B shows the concentrations of propionyl-CoA and methylmalonyl-CoA in primary hepatocytes in the presence of 3 mM ¹³C-ILE. The concentration of propionyl-CoA had an EC₅₀ of 2.04 μM. The concentration of and methylmalonyl-CoA had an EC₅₀ of 1.38 μM. FIG. 2C shows the concentration of propionyl-CoA in primary hepatocytes in the presence of 100 μM ¹³C-heptanoate. The concentration of propionyl-CoA had an EC₅₀ of 3.84 μM. The concentration of methylmalonyl-CoA had an EC₅₀ of 0.02 μM.

FIG. 3 shows representative activity data from PA donor 1 and MMA donor 1 upon treatment of primary hepatocytes with Compound 5. FIG. 3A shows the dose-dependent reduction of propionyl-CoA (“P-CoA”) in PA and MMA primary hepatocytes. FIG. 3B shows the dose-dependent reduction in methylmalonyl (“M-CoA”) labeled with ¹³C in MMA primary hepatocytes. FIG. 3C shows the dose-dependent reduction of propionyl-carnitine (C3) concentration in PA and MMA primary hepatocytes. FIG. 3D shows the dose-dependent reduction of the propionyl-carnitine/acetyl-carnitine (C3/C2) ratio in PA and MMA primary hepatocytes. FIG. 3E shows the dose-dependent reduction of MCA concentration in PA and MMA primary hepatocytes.

FIG. 4 shows dose-response curves for the treatment of PA and MMA primary hepatocytes in static cell culture using from 0.1 μM to 100 μM concentrations of Compound 5. FIG. 4A shows the intracellular concentration of ¹³C—P-CoA in PA and MMA primary hepatocytes treated with Compound 5 under low and high propiogenic conditions. FIG. 4B shows the intracellular concentration of ¹³C-M-CoA in PA and MMA primary hepatocytes treated with Compound 5 under low and high propiogenic conditions. FIG. 4C shows the intracellular concentration of ¹³C-methylmalonic acid in MMA primary hepatocytes treated with Compound 5 under low and high propiogenic conditions.

FIG. 5 shows the pharmacology of Compound 5 in static cell culture in PA primary hepatocytes and MMA primary hepatocytes, under low and high propiogenic conditions. FIG. 5A shows effects of Compound 5 on ¹³C—P-CoA levels measured in PA and MMA pHeps in static cell culture experiments. FIG. 5B shows the effects of Compound 5 on acetyl-CoA levels measured in PA and MMA pHeps in static cell culture experiments. FIG. 5C shows the effects of Compound 5 on CoASH levels measured in PA and MMA pHeps in static cell culture experiments. FIG. 5D shows the dose-dependent increase in Compound 5-CoA formation when PA and MMA pHeps were exposed to Compound 5 for 1.5 h.

FIG. 6 shows the pharmacology of Compound 5 in PA primary hepatocytes, MMA primary hepatocytes, and normal primary hepatocytes. FIG. 6A shows effects of Compound 5 on ¹³C—P-CoA levels measured in PA and MMA pHeps exposed to Compound 5 for 6 days. FIG. 6B shows the effects of Compound 5 on acetyl-CoA levels measured in PA and MMA pHeps exposed to Compound 5 for 6 days. FIG. 6C shows the effect on CoASH levels measured in PA, MMA, and normal pHeps exposed to Compound 5 for 6 days. FIG. 6D shows the dose-dependent increase in Compound 5-CoA formation when PA and MMA pHeps were exposed to Compound 5 for 6 days.

FIG. 7 provides a schematic of the three-part Phase 2 study evaluating Compound 5 in patients with MMA or PA.

FIG. 8 provides a schematic of the 2-period crossover study of Part B of the three-part Phase 2 study of FIG. 7.

### DETAILED DESCRIPTION

### Definitions

Unless otherwise defined, all terms used in this application should be given their standard and typical meanings in the art and are used as those terms would be used by a person of ordinary skill in the art at the time of the invention.

In this application, including the appended claims, the singular forms “a,” “an,” and “the” are often used for convenience. However, it should be understood that these singular forms include the plural unless otherwise specified.

When a numerical range is disclosed herein, it is to be understood that all values and subranges therein are included as if each was expressly disclosed. For example, a range of from about 1 to about 100 is understood to include all values between 1 and 100, e.g., 2, 3, 4, 5, 6, 7, 8, 9, 10, 15, 20, 25, 30, 35, 40, 45, 50, 55, 60, 65, 70, 75, 80, 85, 90, 95, and 99 inclusive of all values and subranges therebetween. As an additional example, a range of from about 1 to about 100 is understood to include all subranges within the range, e.g., 1-42, 37-100, 25-65, 75-98, etc.

The term “pathway” or “metabolic pathway” refers to a series of biochemical or chemical reactions, catalyzed by enzymes that occur within a cell.

The term “metabolite” or variations thereof as used herein refers to molecules which are formed during metabolic processes. The term “metabolite” includes precursors, such as metabolic precursors, of biologically produced molecules and molecules which participate in a biochemical reaction to produce another compound. The term “metabolite” also includes the active moiety formed after administration and catabolism of the compound disclosed herein, e.g., 2-propylpentanoic acid or 2,2-dimethylbutanoic acid. For example, carnitine esters or coenzyme-A esters of 2,2-dimethylbutanoic acid may be formed at various stages of metabolism, and such esters may contribute to the therapeutic effect of the disclosed methods. As such, these metabolites are within the scope of the disclosure.

The phrase “metabolite that accumulates in organic acidemia patients” refers to metabolites that are present in aberrant levels in patients with an organic acidemia. To be clear, the term does not encompass a metabolite that is normally present at non-toxic levels in both healthy and organic acidemia patients. The phrase “metabolite that accumulates in propionic acidemia patients” as used herein refers to a metabolite of one or more of branched chain amino acid, methionine, threonine, odd-chain fatty acids, and cholesterol, wherein abnormal levels of said metabolite (compared to a patient which does not have propionic acidemia) are characteristic of propionic acidemia. Similarly, the phrases “metabolite that accumulates in methylmalonic acidemia patients” and “metabolite that accumulates in propionic acidemia patients” as used herein refers to a metabolite of one or more of a branched chain amino acid, methionine, threonine, odd-chain fatty acids and cholesterol wherein abnormal levels of said metabolite (compared to a patient which does not have methylmalonic or propionic acidemia) are characteristic of methylmalonic acidemia.

The term “compound” as used herein means a molecule which is capable of reducing a particular metabolite associated with metabolic disorders. As used herein, a pharmaceutically acceptable compound includes metabolites, salts, solvates, and prodrugs thereof. For example, any reference to 2,2-dimethylbutyric acid expressly includes prodrugs, metabolites, salts, and solvates of 2,2-dimethylbutyric acid.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods. The term “pharmaceutically acceptable salts” also includes those obtained by reacting the active compound functioning as an acid, with an inorganic or organic base to form a salt, for example salts of ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, and the like. Non limiting examples of inorganic or metal salts include lithium, sodium, calcium, potassium, magnesium salts and the like.

The term “pharmaceutically acceptable esters” include those obtained by replacing a hydrogen on an acidic group with an alkyl group, for example by reacting the acid group with an alcohol or a haloalkyl group. Examples of esters include, but are not limited to, replacing the hydrogen on an —C(O) OH group with an alkyl to form an-C(O) Oalkyl. The alkyl group may be a C₁-C₂₀ straight or branched chain alkyl.

The term “pharmaceutically acceptable solvate” refers to a complex of solute (e.g., active compound, or salt of active compound) and solvent. If the solvent is water, the solvate may be referred to as a hydrate, for example, a mono-hydrate, a di-hydrate, a tri-hydrate, etc.

The term “pharmaceutically acceptable” as described herein is a material that is not biologically or otherwise undesirable, i.e., the material may be incorporated into a pharmaceutical formulation administered to a patient without causing any undesirable biological effects or interacting in a deleterious manner with any of the other components of the composition in which it is contained. When the term “pharmaceutically acceptable” is used to refer to a pharmaceutical excipient, such as a carrier, it is implied that the carrier or excipient has met the required standards of toxicological and manufacturing testing and/or that it is included on the Inactive Ingredient Guide prepared by the U.S. Food and Drug administration.

The term “effective amount” refers to an amount that effective for producing a therapeutic effect upon administration to a subject. The therapeutic effect can include treating a particular disease, such as, but not limited to, achieving a reduction in metabolite levels associated with an organic acidemia.

The term “administering” as used herein includes to any route of administration, for example, oral administration. Administering can also include prescribing a drug to be delivered to a subject, for example, according to a particular dosing regimen, or filling a prescription for a drug that was prescribed to be delivered to a subject, for example, according to a particular dosing regimen.

The terms “treating” and “treatment” include the following actions: (i) preventing a particular disease or disorder from occurring in a subject who may be predisposed to the disease or disorder but has not yet been diagnosed as having it; (ii) curing, treating, or inhibiting the disease, i.e., arresting its development; or (iii) ameliorating the disease by reducing or eliminating symptoms, conditions, and/or by causing regression of the disease.

The terms “patient”, “subject” and “individual” are used interchangeably to refer to a human subject for whom or which therapy is desired, and generally refers to the recipient of the therapy to be practiced according to the present disclosure.

The term “baseline” refers to the levels of a metabolite before the subject was administered with 2,2-dimethylbutyric acid, or an equivalent amount of an ester or pharmaceutically acceptable salt thereof.

The term “control subject” refers to an otherwise similar subject with an organic acidemia (e.g., MMA or PA) that is not treated with 2,2-dimethylbutyric acid, or an equivalent amount of an ester or pharmaceutically acceptable salt thereof. An otherwise similar subject is a subject of the same gender and approximately the same age, weight and disease severity as the subject being treated with 2,2-dimethylbutyric acid, or an equivalent amount of an ester or pharmaceutically acceptable salt thereof.

The terms “once daily administration,” “administered once daily,” or “QD” refers to administration of a compound provided herein at a single dosage amount per day. For example, once daily administration of a 3 mg/kg dose of a compound provided herein means that the subject is administered a single 3 mg/kg dose of said compound a day (i.e., 3 mg/kg total of the compound a day).

The terms “twice daily administration,” “administered twice daily,” or “BID” refers to administration a compound provided herein in equal dosage amounts two times per day. For example, twice daily administration of a 3 mg/kg dose of a compound provided herein means that the subject is administered two 3 mg/kg doses of said compound a day (i.e., 6 mg/kg total of the compound a day).

### Metabolic Disorders

The present disclosure provides for methods of treating particular metabolic disorders that are characterized by the abnormal build-up of toxic metabolites of branched-chain amino acids using an effective amount of 2,2-dimethylbutyric acid or a pharmaceutically acceptable salt or ester thereof. 2,2-dimethylbutyric acid (also referred to 2,2-dimethylbutanoic acid) is represented by the structure (5):

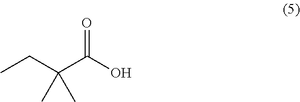

(5) 2,2-dimethylbutyric acid (also known as 2,2-dimethylbutanoic acid or 2,2-dimethylbutyrate; CAS No. 595-37-9).

In some embodiments, the pharmaceutically acceptable salt of 2,2-dimethylbutyric acid is the sodium salt of 2,2-dimethylbutyric acid. 2,2-dimethylbutyric acid sodium salt is represented by the structure (5A).

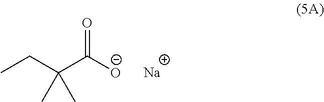

MMA and PA are examples of metabolic disorder that can be treated according to the disclosed methods. PA and MMA are caused by enzyme activity deficiencies that result in the accumulation of metabolites of branched chain amino acids (e.g., valine and isoleucine), methionine, threonine, odd-chain fatty acids, or cholesterol, or combinations thereof. These diseases are classified as an organic acid disorder because patients with these disorders experience an abnormal buildup of organic acids.

PA, an autosomal recessive metabolic disorder, is also known as propionic aciduria, propionyl-CoA carboxylase deficiency, or ketotic glycinemia. The disease is classified as an organic acid disorder which is a condition that leads to an abnormal buildup of particular acids known as organic acids. PA is caused by dysfunction of propionyl-CoA carboxylase (PCC), the heteropolymeric mitochondrial enzyme that catalyzes the conversion of propionyl-CoA to methylmalonyl-CoA. PCC is a heterododecamer (α6β6), comprising six α-subunits and six β-subunits (PCCA and PCCB, respectively). PCC is essential in the normal catabolism of branched-chain amino acids, threonine, methionine, odd-numbered chain length fatty acids, and cholesterol in the body.

PCC enzymatic activity deficiency results in accumulation of propionyl-CoA, propionyl-carnitine, propionyl-glycine, 3-hydroxy propionic acid, 2-methylcitric acid, glycine, ammonia (NH₃ and NH₄⁺) and lactate, among other metabolites in plasma and urine. PCC comprises alpha and beta subunits encoded by PCCA and PCCB, respectively. Different types of mutations can also lead to distinct disease phenotypes. For example, null alleles of PCCA (p.Arg313Ter, p.Ser562Ter) and PCCB (p.Gly94Ter) and several small deletions/insertions and splicing variants are associated with a more severe form of PA. Missense variants, in which partial enzymatic activity is retained (PCCA: p.Ala138Thr, p.Ile164Thr, p.Arg288Gly; PCCB: p.Asn536Asp), are associated with a milder phenotype. Exceptions may include the three PCCB missense variants p.Gly112Asp, p.Arg512Cys, and p.Leu519Pro, which affect heterododecamer formation and are associated with undetectable PCC enzyme activity and the severe phenotype. Other PCCB pathogenic variants such as p.Glu168Lys result in a wide variety of clinical manifestations among affected individuals. Additionally, in some examples, the PCCB pathogenic variant p. Tyr435Cys has been identified in asymptomatic children through newborn screening in Japan. Biallelic mutation of either PCCA or PCCB results in PA. 153 and 138 different types of mutations of PCCA and PCCB are discovered, respectively. For example, one mutation of a subunits of propionyl-CoA carboxylase (PCCA) (c.937C>T/c, 937C>T; pArg313Stop/p.Arg313Stop can result in the loss of the PCCA active site and loss of domains responsible for PCCA interaction with the β subunits of propionyl-CoA carboxylase (PCCB). A non-limiting list of examples of PCCA mutations and PCCB mutations can be found at the following links:
http://cbs.1f1.cuni.cz/pcc/list_of_pcca_mutations.htm
http://cbs.1f1.cuni.cz/pcc/list_of_pccb_mutations.htm, respectively.

The inability to convert propionyl-CoA to methylmalonyl-CoA results in the buildup of certain metabolites, some of which are toxic. The sources of propionyl-CoA include valine, isoleucine, threonine, methionine, odd-chain fatty acids, and cholesterol. The resulting impaired metabolism of these metabolites causes a buildup of metabolites that have deleterious effects on various target organs, e.g., heart, central nervous system etc., considerably shortening the lifespan of affected patients and severely limiting their diet and lifestyle.

Methylmalonic acidemia (MMA) is caused by dysfunction of methylmalonyl-CoA mutase (MM-CoA mutase, or MCM), the mitochondrial enzyme that catalyzes the conversion of methylmalonyl-CoA to succinyl-CoA using adenosylcobalamin (AdoCb1) as a cofactor. The conversion can involve two steps. First step is to convert D-methylmalonyl-CoA to L-methylmalonyl-CoA catalyzed by methylmalonyl-CoA racemase. The second step is to convert L-methylmalonyl-CoA to succinyl-CoA catalyzed by methylmalonyl-CoA mutase. MCM is essential in the normal catabolism of branched-chain amino acids such as leucine and valine as well as methionine, threonine, odd-chain fatty acids and cholesterol. The dysfunction of MCM results in accumulation of methylmalonyl-CoA, methylmalonic acid, as well as the same metabolites that build up in PA listed above. The sources of methylmalonyl-CoA can include, but are not limited to valine, leucine, isoleucine, threonine, methionine, odd-chain fatty acids, and cholesterol.

The failure of properly converting propionyl-CoA to methylmalonyl-CoA, or the failure of properly converting methylmalonyl-CoA to succinyl-CoA, results in accumulation of propionyl-CoA and a derived organic acid, 2-methylcitric acid which disrupts normal Krebs cycle function, also called citric acid cycle or tricarboxylic acid (TCA) cycle. In addition, accumulation of propionyl-CoA results in the inhibition of N-acetylglutamate synthase (NAGS) and consequently lower levels of N-acetylglutamate, resulting in inhibition of urea cycle function (decreased conversion of ammonia to urea) which can lead to hyperammonemia. Together, this metabolic dysregulation leads to the signs and symptoms of PA and MMA.

Therefore, therapeutic strategies which reduce the amount of propionyl-CoA, methylmalonyl-CoA, and/or their related metabolites, and combinations thereof, can be used to treat PA, MMA, as well as other metabolic disorders associated with the production of propionyl-CoA and methylmalonyl-CoA. Non-limiting examples of such metabolic disorders, e.g., disorders involving a BCAA pathway, include isovaleric acidemia, mitochondrial short-chain enoyl-CoA hydratase 1 deficiency (OMIM 616277; ECHS₁ deficiency)), 3-hydroxyisobutyryl-CoA hydrolase deficiency (OMIM 250620; HIBCH deficiency), 3-hydroxyisobutyrate dehydrogenase deficiency, methylmalonate-semialdehyde dehydrogenase deficiency (OMIM 614105), 2-methyl-3-hydroxybutyryl-CoA dehydrogenase deficiency (OMIM 300438; HSD₁₀ deficiency), 2-methylacetoacetyl-CoA thiolase deficiency (OMIM 203750, ACAT1 deficiency), 3-methylcrotonyl-CoA carboxylase deficiency (MCCD), and 3-hydroxy-3-methylglutaric aciduria (HMGD).

Isovaleric acidemia (IVA) is a type of organic acid disorder in which affected individuals have problems breaking down leucine, which results in the accumulation of toxic levels of leucine, 2-ketoisocaproic acid (KICA), isovaleryl-CoA and isovaleric acid. IVA is caused by mutations in the IVD gene and is an autosomal recessive metabolic disorder. Signs and symptoms may range from very mild to life-threatening. In severe cases, symptoms begin within a few days of birth and include poor feeding, vomiting, seizures, and lack of energy (lethargy); these may progress to more serious medical problems including seizures, coma, and possibly death. In other cases, signs and symptoms appear during childhood and may come and go over time. A characteristic sign of IVA is a distinctive odor of sweaty feet during acute illness. Other features may include failure to thrive or delayed development.

Mitochondrial short-chain enoyl-CoA hydratase 1 deficiency (ECHS1D; OMIM 616277) is caused by a dysfunction of short-chain enoyl-CoA hydratase (ECHS1; EC 4.2.1.17; formerly called SCEH). ECHS1 is a mitochondrial enzyme that catalyzes the conversion of unsaturated trans-2-enoyl-CoA species to their corresponding 3 (S)-hydroxyacyl-CoA species. ECHS1 is essential for the normal catabolism of the branched-chain amino acids, isoleucine, and valine, and also functions in the β-oxidation of short- and medium-chain fatty acids. The clinical phenotype of ECHS1 deficiency is not consistent with that of a fatty acid oxidation disorder, suggesting that this is primarily a disorder of branched-chain amino acid metabolism. ECHS1 deficiency is characterized by the accumulation of abnormal metabolites including: S-(2-carboxypropyl) cysteine, S-(2-carboxypropyl) cysteamine, N-acetyl-S-(2-carboxypropyl) cysteine, S-(2-carboxypropyl) cysteine carnitine, methacrylylglycine, S-(2-carboxyethyl) cysteine, S-(2-carboxyethyl) cysteamine, N-acetyl-S-(2-carboxyethyl) cysteine and 2,3-dihydroxy-2-methylbutyric acid. Therefore, therapeutic strategies which reduce the production of the above metabolites can be used to treat mitochondrial short-chain enoyl-CoA hydratase 1 deficiency.

Methylacrylic aciduria (OMIM 250620; also called 3-hydroxyisobutyryl-CoA hydrolase deficiency) is caused by dysfunction of 3-hydroxyisobutyryl-CoA hydrolase (HIBCH; EC 3.1.2.4), the mitochondrial enzyme that catalyzes the conversion of 3-hydroxyisobutyryl-CoA to free 3-hydroxyisobutyrate. HIBCH is essential in the normal catabolism of the branched-chain amino acid valine. HIBCH is also reactive towards 3-hydroxypropionyl-CoA, giving it a dual role in a secondary pathway of propionate metabolism. The sources of hydroxypropionyl-CoA can include, but are not limited to valine, leucine, isoleucine, threonine, methionine, odd-chain fatty acids and cholesterol. HIBCH deficiency results in the accumulation of abnormal metabolites including: (S)-3-hydroxyisobutyryl-L-carnitine, S-(2-carboxypropyl) cysteine, S-(2-carboxypropyl) cysteamine, N-acetyl-S-(2-carboxypropyl) cysteine, S-(2-carboxypropyl) cysteine carnitine, methacrylylglycine, S-(2-carboxyethyl) cysteine, S-(2-carboxyethyl) cysteamine, N-acetyl-S-(2-carboxyethyl) cysteine and 2,3-dihydroxy-2-methylbutyric acid. Therefore, therapeutic strategies which reduce the production of the above metabolites can be used to treat methylacrylic aciduria.

3-hydroxyisobutyrate dehydrogenase (HIBADH; EC1.1.1.31) deficiency may be caused by mutations in the HIBADH gene, encoding an enzyme that catalyzes the NAD (+)-dependent, reversible oxidation of 3-hydroxyisobutyrate to methylmalonate semialdehyde, although no mutations have been identified as causing this disease. 3-hydroxyisobutyrate dehydrogenase deficiency may also be caused by defects in respiratory chain function such as Leigh's syndrome. HIBADH is essential in the normal catabolism of the branched-chain amino acid valine. HIBADH deficiency is one cause of 3-hydroxyisobutyric aciduria, a disorder with a heterogeneous clinical phenotype that can also be caused by defects in the electron transport chain or by methylmalonate semialdehyde dehydrogenase deficiency. The dysfunction of HIBADH has been shown to result in accumulation of 3-hydroxyisobutyrate and 3-hydroxyisobutyryl carnitine. Therefore, therapeutic strategies which reduce production of the above metabolites can be used to treat 3-hydroxyisobutyrate dehydrogenase deficiency.

Methylmalonate semialdehyde dehydrogenase deficiency (MMSDHD; OMIM 614105) is caused by the deficiency of the enzyme methylmalonate semialdehyde dehydrogenase (MMSDH; EC 1.2.1.27). MMSDH is encoded by the ALDH6A1 gene and catalyzes the oxidative decarboxylation of methylmalonate semialdehyde into propionyl-CoA. MMSDH is essential in the normal catabolism of the branched-chain amino acid valine and thymine metabolism. MMSDH deficiency is one cause of 3-hydroxyisobutyric aciduria, a disorder with a heterogeneous clinical phenotype that can also be caused by defects in the electron transport chain or by 3-hydroxyisobutyrate dehydrogenase (HIBADH) deficiency. The dysfunction of MMSDH has been shown to result in accumulation of 3-hydroxyisobutyrate and 3-hydroxyisobutyryl carnitine, as well as 3-hydroxypropionic acid and 2-ethyl-3-hydroxypropionic acid. Therefore, therapeutic strategies which reduce production of the above metabolites can be used to treat methylmalonate semialdehyde dehydrogenase deficiency.

17-β hydroxysteroid dehydrogenase X deficiency (OMIM 300438) is caused by the deficiency of hydroxysteroid 17-β dehydrogenase 10 (EC 1.1.1.178; also known as 2-methyl-3-hydroxybutyryl-CoA dehydrogenase or 3-hydroxyacyl-CoA dehydrogenase type II). Hydroxysteroid 17-β dehydrogenase 10 (HSD10) is a multifunctional mitochondrial enzyme that catalyzes the reversible conversion of 2-methyl-3-hydroxybutyryl-CoA to 2-methylacetoacetyl-CoA and is an essential enzyme in the degradation pathway of isoleucine. HSD10 is encoded by the gene HSD17B10 (formerly known as HADH2) and HSD10 deficiency is caused by mutations in the HSD17B10 gene. This syndrome has a biochemical phenotype similar to that of β-ketothiolase deficiency, but represents a unique disorder which typically shows a more severe clinical phenotype. HSD10 is known to catalyze the oxidation of a wide variety of steroid receptor modulators and thus plays a role in sex steroid and neuroactive steroid metabolism, and is also a subunit of mitochondrial ribonuclease P which is involved in tRNA maturation. The dysfunction of HSD 10 in isoleucine degradation has been shown to result in the accumulation of tiglylglycine, 2-methyl-3-hydroxybutyrate, OH—C5 carnitine, and in some cases 2-ethylhydracrylic acid, 3-hydroxyisobutyrate and tiglylglutamic acid. Therefore, therapeutic strategies which reduce production of the above metabolites can be used to treat 17-β hydroxysteroid dehydrogenase X deficiency.

Alpha-methylacetoacetic aciduria (OMIM 203750) is caused by the deficiency of 3-methylacetoacetyl-CoA thiolase (EC 2.3.1.9; more commonly called β-ketothiolase or T2). β-ketothiolase (β-KT) is a K⁺-dependent mitochondrial enzyme that catalyzes the thiolytic cleavage of 2-methylacetoacetyl-CoA to produce acetyl-CoA and propionyl-CoA. β-KT is an essential enzyme in the degradation pathway of isoleucine. β-KT is encoded by the gene ACAT1 and β-KT deficiency is caused by mutations in the ACAT1 gene. This syndrome has a biochemical phenotype similar to that of HSD10 deficiency but represents a unique disorder as blockade of isoleucine degradation by loss of β-KT does not commonly cause developmental disabilities except for a few cases with neurological sequelae attributed to severe ketoacidotic attacks. The dysfunction of β-KT in isoleucine degradation has been shown to result in the accumulation of ketones such as 3-hydroxybutyrate, acetoacetic acid, 2-methylacetoacetic acid and 2-butanone, as well as tiglylglycine, 2-methyl-3-hydroxybutyrate, OH—C5 carnitine, and in some cases 2-ethylhydracrylic acid, 3-hydroxyisobutyrate and tiglylglutamic acid. Therefore, therapeutic strategies which reduce production of the above metabolites can be used to treat alpha-methylacetoacetic aciduria.

Other non-limiting examples of CoA disorders that can be treated by the presently disclosed methods include glutaric aciduria type I, long-chain acyl-CoA dehydrogenase deficiency (LCHAD), very-long chain acyl-CoA dehydrogenase deficiency (VLCAD), and Refsum Disease and the diseases in Table 1.

TABLE 1
Additional Diseases That Can Be Treated According to the Disclosed Methods
Disease Name

| | | |
|---|---|---|
| Acromicric dysplasia | Barth syndrome | cytochrome P450 family 17 |
| | | subfamily A member 1 |
| aminoacylase 1 | Inherited thyroxine-binding | acyl-CoA dehydrogenase very |
| | globulin deficiency | long chain |
| Caffey disease | glycyl-tRNA synthetase | L-2-hydroxyglutarate |
| | | dehydrogenase |
| carbonic anhydrase 5A | acyl-CoA dehydrogenase | acyl-CoA oxidase 1 |
| | short/branched chain | |
| collagen type IX alpha 1 | AU RNA binding | metabolism of cobalamin |
| chain | methylglutaconyl-CoA | associated C |
| | hydratase | |
| collagen type IX alpha 3 | hydroxyacyl-CoA | Aminoacylase 1 deficiency |
| chain | dehydrogenase trifunctional | |
| | multienzyme complex subunit | |
| | alpha | |
| hydroxyacyl-CoA | 3-oxoacid CoA-transferase 1 | Short/branched chain acyl- |
| dehydrogenase trifunctional | | CoA dehydrogenase deficiency |
| multienzyme complex | | |
| subunit beta | | |
| Cowden syndrome | platelet activating factor | Dilated cardiomyopathy with |
| | acetylhydrolase 1b regulatory | ataxia syndrome |
| | subunit 1 | |
| Myoclonus-dystonia | hydroxyacyl-CoA | heparan-alpha-glucosaminide |
| | dehydrogenase | N-acetyltransferase |
| retinoic acid receptor alpha | peroxisomal biogenesis factor | fatty acid 2-hydroxylase |
| | 7 | |
| triosephosphate isomerase 1 | hydroxysteroid 17-beta | ACAD9 deficiency |
| | dehydrogenase 4 | |
| Medium-chain acyl-CoA | phytanoyl-CoA 2-hydroxylase | Isobutyryl-CoA dehydrogenase |
| dehydrogenase deficiency | | deficiency |
| Short-chain acyl-CoA | Desmosterolosis | hepatic and glial cell adhesion |
| dehydrogenase deficiency | | molecule |
| Very long-chain acyl-CoA | TGFB induced factor | metabolism of cobalamin |
| dehydrogenase deficiency | homeobox 1 | associated D |
| Cytochrome P450 | ATP binding cassette | Fatty acid hydroxylase- |
| oxidoreductase deficiency | subfamily D member 4 | associated neurodegeneration |
| 3-beta-hydroxysteroid | cytochrome P450 family 7 | LMBR1 domain containing 1 |
| dehydrogenase deficiency | subfamily B member 1 | |
| 17 alpha-hydroxylase/17,20- | Ohdo syndrome, Say-Barber- | tyrosine aminotransferase |
| lyase deficiency | Biesecker-Young-Simpson | |
| | variant | |
| Beta-ketothiolase deficiency | arachidonate 12-lipoxygenase, | GABA-transaminase |
| | 12R type | deficiency |
| Cerebrotendinous | Intervertebral disc disease | 3-hydroxy-3-methylglutaryl- |
| xanthomatosis | | CoA lyase |
| DOORS syndrome | aldo-keto reductase family 1 | acyl-CoA synthetase family |
| | member D1 | member 3 |
| Dopamine beta-hydroxylase | acyl-CoA dehydrogenase | Combined malonic and |
| deficiency | family member 8 | methylmalonic aciduria |
| 3-hydroxyacyl-CoA | histone deacetylase 4 | Alpha-methylacyl-CoA |
| dehydrogenase deficiency | | racemase deficiency |
| Glutaric acidemia type I | serine palmitoyltransferase | MEGDEL syndrome |
| | long chain base subunit 1 | |
| Glutaric acidemia type II | pyruvate dehydrogenase | CLPB deficiency |
| | phosphatase catalytic subunit | |
| | 1 | |
| propionyl-CoA carboxylase | Propionic acidemia | Multiple epiphyseal dysplasia |
| subunit alpha | | |
| propionyl-CoA carboxylase | Genitopatellar syndrome | collagen type IX alpha 2 chain |
| subunit beta | | |
| Glycogen storage disease | solute carrier family 25 | Hereditary multiple |
| type V | member 19 | osteochondromas |
| Congenital bile acid | cytochrome P450 family 27 | lysine acetyltransferase 6B |
| synthesis defect type 2 | subfamily A member 1 | |
| aminomethyltransferase | malonyl-CoA decarboxylase | Stickler syndrome |
| dihydrolipoamide | acyl-CoA dehydrogenase | Saethre-Chotzen syndrome |
| dehydrogenase | short chain | |
| Isovaleric acidemia | acyl-CoA dehydrogenase | Corticosterone methyloxidase |
| | medium chain | deficiency |
| Succinyl-CoA:3-ketoacid | isovaleryl-CoA | 3-methylcrotonyl-CoA |
| CoA transferase deficiency | dehydrogenase | carboxylase deficiency |
| 3-hydroxy-3-methylglutaryl- | Amish lethal microcephaly | pyruvate dehydrogenase E1 |
| CoA lyase deficiency | | beta subunit |
| Dihydrolipoamide | arachidonate lipoxygenase 3 | Tyrosinemia |
| dehydrogenase deficiency | | |
| Malonyl-CoA decarboxylase | metabolism of cobalamin | Methylmalonic acidemia with |
| deficiency | associated A | homocystinuria |
| 3-methylglutaconyl-CoA | metabolism of cobalamin | Methylmalonic acidemia |
| hydratase deficiency | associated B | |
| Mitochondrial complex I | acetyl-CoA acetyltransferase 1 | hydroxysteroid 17-beta |
| deficiency | | dehydrogenase 10 |
| Costeff syndrome | methylmalonyl-CoA | pyruvate dehydrogenase E1 |
| | epimerase | alpha 1 subunit |
| D-bifunctional protein | pyruvate dehydrogenase | Pyruvate dehydrogenase |
| deficiency | complex component X | deficiency |
| Peroxisomal acyl-CoA | dihydrolipoamide S- | Distal hereditary motor |
| oxidase deficiency | acetyltransferase | neuropathy, type V |
| Succinic semialdehyde | glutaryl-CoA dehydrogenase | Auriculo-condylar syndrome |
| dehydrogenase deficiency | | |
| host cell factor C1 | methylcrotonoyl-CoA | Megalencephalic |
| | carboxylase 1 | leukoencephalopathy with |
| | | subcortical cysts |
| EBP, cholestenol delta- | methylcrotonoyl-CoA | alpha-methylacyl-CoA |
| isomerase | carboxylase 2 | racemase |
| myotubularin 1 | Long-chain 3-hydroxyacyl- | 4-hydroxyphenylpyruvate |
| | CoA dehydrogenase | dioxygenase |
| | deficiency | |
| HSD10 disease | biotinidase | 2-hydroxyglutaric aciduria |
| X-linked sideroblastic | methylmalonyl-CoA mutase | |
| anemia | | |

In some embodiments, the present disclosure provides methods of treating one or more organic acidemias disclosed herein by administering an effective amount of 2,2-dimethylbutyric acid, or a pharmaceutically acceptable salt or ester thereof. In some embodiments, the effective amount of 2,2-dimethylbutyric acid, or pharmaceutically acceptable salt or ester thereof, reduces the formation and/or amount of metabolites associated with the organic acidemia. In particular embodiments, the present disclosure provides for methods of reducing isovaleryl-CoA, propionyl-CoA and/or methylmalonyl-CoA production in a subject. In some embodiments, the present disclosure provides methods for treating IVA, PA, and MMA, thereby addressing key needs in the fields of metabolic disorder therapeutics.

In some embodiments, the level of a metabolite that is associated with organic acidemia patients (e.g., isovaleryl-CoA, propionyl-CoA or methylmalonyl-CoA), is reduced by at least about 1% to about 100%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99%, inclusive of all values and subranges therebetween, compared to baseline or a control. For example, the reduced level may be at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%. In some embodiments, the at least one metabolite comprises 2-ketoisocaproate, isovaleryl-CoA, 3-methylcrotonyl-CoA, 3-methylglutaconyl-CoA, 3-OH-3-methylglutaryl-CoA, 2-keto-3-methylvalerate, 2-methylbutyryl-CoA, tiglyl-CoA, 2-methyl-3-OH-butyryl-CoA, 2-methyl-acetoacetyl-CoA, 2-ketoisovalerate, isobutyryl-CoA, methylacrylyl-CoA, 3-OH-isobutyryl-CoA, 3-OH-isobutyrate, methylmalonic semialdehyde, propionyl-CoA, or methylmalonyl-CoA, or combinations thereof. In other embodiments, the at least one metabolite comprises propionic acid, 3-hydroxypropionic acid, methylcitrate, glycine, or propionylcarnitine, or combinations thereof.

As discussed herein, in embodiments, the inhibitor is 2,2-dimethylbutyric acid (represented by structure 5) or a pharmaceutically acceptable salt thereof (e.g., the sodium salt, represented by structure 5A).

In some embodiments, the present disclosure provides a method of treating a patient with 2,2-dimethylbutyric acid or pharmaceutically acceptable salt thereof (e.g., a sodium salt) that is biotransformed into 2,2-dimethylbutyryl-CoA in vivo. In some embodiments, the method comprises treating a patient with a compound 2,2-dimethylbutyric acid or pharmaceutically acceptable salt thereof that forms 2,2-dimethylbutyryl-CoA in an intracellular compartment

Without being bound by theory, the compounds of the present disclosure can be administered as a free acid or a pharmaceutically acceptable salt, and the compound can be converted (i.e., metabolized) in vivo to form one or more therapeutically active metabolites that effectively treat the diseases disclosed herein, e.g., PA and MMA. In some embodiments, the metabolites of 2,2-dimethylbutyric acid suitable for use in the disclosed methods include 2,2-dimethylbutyryl-CoA and 2,2-dimethylbutyryl-carnitine.

The structure of 2,2-dimethylbutyryl-carnitine is provided below:

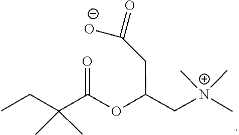 [SMILES: CCC(C)(C)C(=O)OC(CC(=O)[O-])C[N+](C)(C)C]

In some embodiments, the 2,2-dimethylbutyryl-carnitine is 2,2-dimethylbutyryl-L-carnitine having the structure:

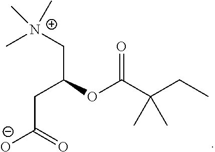 [SMILES: CCC(C)(C)C(=O)O[C@@H](CC(=O)[O-])C[N+](C)(C)C]

The structure of 2,2-dimethylbutyryl-CoA is provided below:

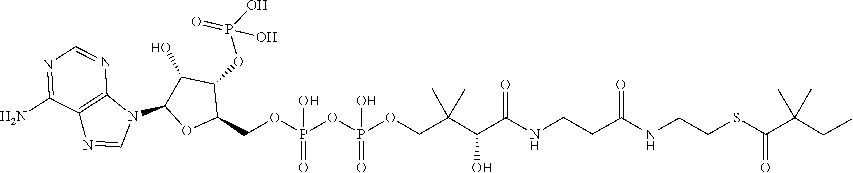 [SMILES: CCC(C)(C)C(=O)SCCNC(=O)CCNC(=O)[C@H](O)C(C)(C)COP(=O)(O)OP(=O)(O)OC[C@H]1O[C@@H](n2cnc3c(N)ncnc32)[C@H](O)[C@@H]1OP(=O)(O)O]

Blood plasma concentrations for sodium 2,2-dimethylbutyrate, or an equivalent dose of 2,2-dimethylbutyric acid or a different pharmaceutically acceptable salt, thereof following administration was dose proportional over a dose range of 1 mg/kg to 30 mg/kg, for example, about 1 mg/kg, about 3 mg/kg, about 9 mg/kg, about 10 mg/kg, and about 15 mg/kg, administered either once or twice daily (see, e.g., Example 9).

In some embodiments, after orally administering a total daily dose ranging from 1-30 mg/kg of sodium 2,2-dimethylbutyrate, or an equivalent dose of 2,2-dimethylbutyric acid or a different pharmaceutically acceptable salt thereof, the method provides at least one of the following pharmacokinetic characteristics:
(a) an average maximum steady state blood plasma concentration (Cmax) ranging from about 1 μg/mL to about 500 μg/mL;
(b) an average steady state blood plasma concentration curve from 0-12h (AUC₀₋₁₂ₕ) ranging from about 45 h*μg/mL to about 4000 h*μg/mL; and/or
(c) an average steady state blood plasma concentration curve from 0-24h (AUC₀₋₂₄ₕ) ranging from about 20 h*μg/mL to about 5000 h*μg/mL.

In some embodiments, following oral administration of a total daily dose ranging from 1-30 mg/kg of sodium 2,2-dimethylbutyrate, or an equivalent dose of 2,2-dimethylbutyric acid or a different pharmaceutically acceptable salt thereof, the average steady state Cmax ranging from about 1 μg/mL to about 500 μg/mL, for example, about 1 μg/mL, about 2 μg/mL, about 3 μg/mL, about 4 μg/mL, about 4.5 μg/mL, about 5 μg/mL, about 5.5 μg/mL, about 6 μg/mL, about 6.5 μg/mL, about 7 μg/mL, about 7.5 μg/mL, about 8 μg/mL, about 8.5 μg/mL, about 8.5 μg/mL, about 9 μg/mL, about 9.5 μg/mL, about 10 μg/mL, 10.5 μg/mL, about 11 μg/mL, about 11.5 μg/mL, about 12 μg/mL, about 12.5 μg/mL, about 13 μg/mL, about 13.5 μg/mL, about 14 μg/mL, about 14.5, about 15 μg/mL, about 20 μg/mL, about 30 μg/mL, about 40 μg/mL, about 50 μg/mL, about 60 μg/mL, about 70 μg/mL, about 80 μg/mL, about 90 μg/mL, about 100 μg/mL, about 110 μg/mL, about 115 μg/mL, about 120 μg/mL, about 125 μg/mL, about 130 μg/mL, about 131 μg/mL, about 135 μg/mL, about 140 μg/mL, about 145 μg/mL, about 150 μg/mL, about 155 μg/mL, about 160 μg/mL, about 165 μg/mL, about 170 μg/mL, about 185 μg/mL, about 190 μg/mL, about 195 μg/mL, about 200 μg/mL, about 205 μg/mL, about 210 μg/mL, about 215 μg/mL, about 220 μg/mL, about 225 μg/mL, about 230 μg/mL, about 235 μg/mL, about 240 μg/mL, about 245 μg/mL, about 250 μg/mL, about 255 μg/mL, about 260 μg/mL, about 265 μg/mL, about 270 μg/mL, about 285 μg/mL, about 290 μg/mL, about 295 μg/mL, about 300 μg/mL, about 305 μg/mL, about 310 μg/mL, about 315 μg/mL, about 320 μg/mL, about 325 μg/mL, about 330 μg/mL, about 335 μg/mL, about 340 μg/mL, about 345 μg/mL, about 350 μg/mL, about 355 μg/mL, about 360 μg/mL, about 365 μg/mL, about 370 μg/mL, about 385 μg/mL, about 390 μg/mL, about 395 μg/mL, about 400 μg/mL, about 405 μg/mL, about 410 μg/mL, about 415 μg/mL, about 420 μg/mL, about 425 μg/mL, about 430 g/mL, about 435 μg/mL, about 440 μg/mL, about 445 μg/mL, about 450 μg/mL, about 455 μg/mL, about 460 μg/mL, about 465 μg/mL, about 470 μg/mL, about 485 μg/mL, about 490 μg/mL, about 495 μg/mL, or about 500 μg/mL, including all ranges and values therebetween. In some embodiments, the average steady state Cmax ranges from about 1 μg/mL to about 225 μg/mL. In some embodiments, the Cmax (% CV) may be 80-125% of any of the above values or ranges of the above values. In some embodiments, the Cmax (% CV) ranges from about 80-125% of about 3.67 (28) μg/mL following once daily administration of about 1 mg/kg of sodium 2,2-dimethylbutyrate. In some embodiments, the Cmax (% CV) ranges from about 80-125% of about 13.6 (28) μg/mL following once daily administration of about 3 mg/kg of sodium 2,2-dimethylbutyrate. In some embodiments, the Cmax (% CV) ranges from about 80-125% of about 50.7 (28) μg/mL following once daily administration of about 10 mg/kg of sodium 2,2-dimethylbutyrate. In some embodiments, the Cmax (% CV) ranges from about 80-125% of about 26.3 (28) μg/mL following twice daily administration of about 3 mg/kg mg/kg of sodium 2,2-dimethylbutyrate (for a total daily dose of 6 mg/kg). In some embodiments, the Cmax (% CV) ranges from about 80-125% of about 78.8 (28) μg/mL following twice daily administration of about 9 mg/kg mg/kg of sodium 2,2-dimethylbutyrate (for a total daily dose of 18 mg/kg). In some embodiments, the Cmax (% CV) ranges from about 80-125% of about 131 (28) μg/mL following twice daily administration of about 15 mg/kg mg/kg of sodium 2,2-dimethylbutyrate (for a total daily dose of 30 mg/kg).

In some embodiments, following oral administration of a total daily dose ranging from 1-30 mg/kg of sodium 2,2-dimethylbutyrate, or an equivalent dose of 2,2-dimethylbutyric acid or a different pharmaceutically acceptable salt thereof, the average AUC₀₋₁₂ₕ in the subject ranges from about 45 h*μg/mL to about 4000 h*μg/mL, for example, about 45 h*μg/mL, about 55 h*μg/mL, about 60 h*μg/mL, about 70 h*μg/mL, about 80 h*μg/mL, about 90 h*μg/mL, about 100 h*μg/mL, about 150 h*μg/mL, about 200 h*μg/mL, about 300 h*μg/mL, about 400 h*μg/mL, about 500 h*μg/mL, about 600 h*μg/mL, about 700 h*μg/mL, about 800 h*μg/mL, about 900 h*μg/mL, about 1000 h*μg/mL, about 1100 h*μg/mL, about 1200 h*μg/mL, about 1300 h*μg/mL, about 1400 h*μg/mL, about 1500 h*μg/mL, about 1600 h*μg/mL, about 1700 h*μg/mL, about 1800 h*μg/mL, about 1900 h*μg/mL, about 2000 h*μg/mL, about 2100 h*μg/mL, about 2200 h*μg/mL, about 2300 h*μg/mL, about 2400 h*μg/mL, about 2500 h*μg/mL, about 2600 h*μg/mL, about 2700 h*μg/mL, about 2800 h*μg/mL, about 2900 h*μg/mL, about 3000 h*μg/mL, about 3100 h*μg/mL, about 3200 h*μg/mL, about 3300 h*μg/mL, about 3400 h*μg/mL, about 3500 h*μg/mL, about 3600 h*μg/mL, about 3700 h*μg/mL, about 3800 h*μg/mL, about 3900 h*μg/mL, or about 4000 h*μg/mL, including all ranges and values therebetween. In some embodiments, the AUC₀₋₁₂ₕ ranges from about 45 h*μg/mL to about 2000 h*μg/mL. In some embodiments, the AUC₀₋₁₂ₕ may be 80-125% of any of the above values or ranges of the above values. In some embodiments, the AUC₀₋₁₂ₕ (CV %) ranges from about 80-125% of about 238 (23) h*μg/mL following twice daily administration of about 3 mg/kg of sodium 2,2-dimethylbutyrate (for a total daily dose of 6 mg/kg). In some embodiments, the AUC₀₋₁₂ₕ (CV %) ranges from about 80-125% of about 715 (23) h*μg/mL following twice daily administration of about 9 mg/kg mg/kg of sodium 2,2-dimethylbutyrate (for a total daily dose of 18 mg/kg). In some embodiments, the AUC₀₋₁₂ₕ (CV %) ranges from about 80-125% of about 1190 (23) h*μg/mL following twice daily administration of about 15 mg/kg mg/kg of sodium 2,2-dimethylbutyrate (for a total daily dose of 30 mg/kg).

In some embodiments, following oral administration of a total daily dose ranging from 1-30 mg/kg of sodium 2,2-dimethylbutyrate, or an equivalent dose of 2,2-dimethylbutyric acid or a different pharmaceutically acceptable salt thereof, the method provides and average AUC₀₋₂₄ₕ ranging from about 20 h*μg/mL to about 5000 h*μg/mL, for example, about 20 h*μg/mL, about 25 h*μg/mL, about 35 h*μg/mL, about 45 h*μg/mL, about 55 h*μg/mL, about 60 h*μg/mL, about 70 h*μg/mL, about 80 h*μg/mL, about 90 h*μg/mL, about 100 h*μg/mL, about 150 h*μg/mL, about 200 h*μg/mL, about 300 h*μg/mL, about 400 h*μg/mL, about 500 h*μg/mL, about 600 h*μg/mL, about 700 h*μg/mL, about 800 h*μg/mL, about 900 h*μg/mL, about 1000 h*μg/mL, about 1100 h*μg/mL, about 1200 h*μg/mL, about 1300 h*μg/mL, about 1400 h*μg/mL, about 1500 h*μg/mL, about 1600 h*μg/mL, about 1700 h*μg/mL, about 1800 h*μg/mL, about 1900 h*μg/mL, about 2000 h*μg/mL, about 2100 h*μg/mL, about 2200 h*μg/mL, about 2300 h*μg/mL, about 2400 h*μg/mL, about 2500 h*μg/mL, about 2600 h*μg/mL, about 2700 h*μg/mL, about 2800 h*μg/mL, about 2900 h*μg/mL, about 3000 h*μg/mL, about 3100 h*μg/mL, about 3200 h*μg/mL, about 3300 h*μg/mL, about 3400 h*μg/mL, about 3500 h*μg/mL, about 3600 h*μg/mL, about 3700 h*μg/mL, about 3800 h*μg/mL, about 3900 h*μg/mL, or about 4000 h*μg/mL, about 4100 h*μg/mL, about 4200 h*μg/mL, about 4300 h*μg/mL, about 4400 h*μg/mL, about 4500 h*μg/mL, about 4600 h*μg/mL, about 4700 h*μg/mL, about 4800 h*μg/mL, about 4900 h*μg/mL, or about 5000 h*μg/mL, including all ranges and values therebetween. In some embodiments, the method provides an average AUC₀₋₂₄ₕ ranging from about 20 h*μg/mL to about 3000 h*μg/mL. In some embodiments, the AUC₀₋₂₄ₕ may be 80-125% of any of the above values or ranges of the above values. In some embodiments, the AUC₀₋₂₄ₕ (CV %) ranges from about 80-125% of about 47 (23) h*μg/mL following once daily administration of about 1 mg/kg of sodium 2,2-dimethylbutyrate. In some embodiments, the AUC₀₋₁₂ₕ (CV %) ranges from about 80-125% of about 152 (23) h*μg/mL following once daily administration of about 3 mg/kg mg/kg of sodium 2,2-dimethylbutyrate. In some embodiments, the AUC₀₋₂₄ₕ (CV %) ranges from about 80-125% of about 618 (23) h*μg/mL following once daily administration of about 10 mg/kg mg/kg of sodium 2,2-dimethylbutyrate.

In some embodiments, following oral administration of a total daily dose ranging from 1-30 mg/kg of sodium 2,2-dimethylbutyrate, or an equivalent dose of 2,2-dimethylbutyric acid or a different pharmaceutically acceptable salt thereof, the subject has a steady state blood plasma concentration at 12 h (C12) ranging from about 1 μg/mL to about 150 μg/mL, for example, about 1 h μg/mL, about 2 μg/mL, about 5 μg/mL, about 10 μg/mL, about 15 μg/mL, about 20 μg/mL, about 25 μg/mL, about 30 μg/mL, about 35 μg/mL, about 40 μg/mL, about 45 μg/mL, about 50 μg/mL, about 55 μg/mL, about 60 μg/mL, about 65 μg/mL, about 70 μg/mL, about 75 μg/mL, about 80 μg/mL, about 85 μg/mL, about 90 μg/mL, about 95 μg/mL, about 100 μg/mL, about 105 μg/mL, about 110 μg/mL, about 115 μg/mL, about 120 μg/mL, about 125 μg/mL, about 130 μg/mL, about 135 μg/mL, about 140 μg/mL, about 145 μg/mL, or about 150 μg/mL, including all ranges and values therebetween. In some embodiments, the C12 may be 80-125% of any of the above values or ranges of the above values. In some embodiments, the C12 (CV %) ranges from about 80-125% of about 11.6 (77) μg/mL following twice daily administration of about 3 mg/kg of sodium 2,2-dimethylbutyrate (for a total daily dose of 6 mg/kg). In some embodiments, the C12 (CV %) ranges from about 80-125% of about 34.7 (77) μg/mL following twice daily administration of about 9 mg/kg mg/kg of sodium 2,2-dimethylbutyrate (for a total daily dose of 18 mg/kg). In some embodiments, the C12 (CV %) ranges from about 80-125% of about 57.9 (77) μg/mL following twice daily administration of about 15 mg/kg mg/kg of sodium 2,2-dimethylbutyrate (for a total daily dose of 30 mg/kg).

In some embodiments, following oral administration of a total daily dose ranging from 1-30 mg/kg of sodium 2,2-dimethylbutyrate, or an equivalent dose of 2,2-dimethylbutyric acid or a different pharmaceutically acceptable salt thereof, the method provides an average steady state blood plasma concentration at 24 h (C24) in a subject ranging from about 0.5 μg/mL to about 50 μg/mL, for example, about 0.5 μg/mL, about 1 h μg/mL, about 1.5 μg/mL, about 2 μg/mL, about 2.5 μg/mL, about 3 μg/mL, about 3.5 μg/mL, about 4 μg/mL, about 4.5 μg/mL, about 5 μg/mL, about 5.5 μg/mL, about 6 μg/mL, about 6.5 μg/mL, about 7 μg/mL, about 7.5 μg/mL, about 8 μg/mL, about 8.5 μg/mL, about 9 μg/mL, about 9.5 μg/mL, about 10 μg/mL, about 10.5 μg/mL, about 11 μg/mL, about 11.5 μg/mL, about 12 μg/mL, about 12.5 μg/mL, about 13 μg/mL, about 13.5 μg/mL, about 14 μg/mL, about 14.5 μg/mL, about 15 μg/mL, about 15.5 μg/mL, about 16 μg/mL, about 16.5 μg/mL, about 17 μg/mL, about 17.5 μg/mL, about 18 μg/mL, about 18.5 μg/mL, about 19 μg/mL, about 19.5 μg/mL, about 20 μg/mL, about 30 μg/mL, about 35 μg/mL, about 40 μg/mL, about 45 μg/mL, about 50 μg/mL, including all ranges and values therebetween. In some embodiments, the C₂₄ may be 80-125% of any of the above values or ranges of the above values. In some embodiments, the C₂₄ (CV %) ranges from about 80-125% of about 0.97 (77) μg/mL following once daily administration of about 1 mg/kg of sodium 2,2-dimethylbutyrate. In some embodiments, the C₂₄ (CV %) ranges from about 80-125% of about 2.2 (77) μg/mL following once daily administration of about 3 mg/kg mg/kg of sodium 2,2-dimethylbutyrate. In some embodiments, the C₂₄ (CV %) ranges from about 80-125% of about 11 (77) μg/mL following once daily administration of about 10 mg/kg mg/kg of sodium 2,2-dimethylbutyrate.

In some embodiments, the methods of the disclosure comprise administering a compound disclosed herein (e.g., sodium 2,2-dimethylbutanoate, or an equivalent dose of 2,2-dimethylbutyric acid, or an CoA ester or carnitine ester thereof, or a pharmaceutically acceptable salt, or ester thereof), in an amount ranging from about 1 mg/kg to about 50 mg/kg, e.g., about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, or about 50 mg/kg, including all ranges and values therebetween. In some embodiments, the sodium 2,2-dimethylbutanoate, or an equivalent dose of 2,2-dimethylbutyric acid or a CoA ester or carnitine ester thereof, or a pharmaceutically acceptable salt or ester thereof is administered in an amount ranging from about 1 mg/kg to about 3 mg/kg. In some embodiments, the sodium 2,2-dimethylbutanoate, or an equivalent dose of 2,2-dimethylbutyric acid or a CoA ester or carnitine ester thereof, or a pharmaceutically acceptable salt or ester thereof is administered in an amount ranging from about 1 mg/kg to about 9 mg/kg. In some embodiments, the sodium 2,2-dimethylbutanoate, or an equivalent dose of 2,2-dimethylbutyric acid or a CoA ester or carnitine ester thereof, or a pharmaceutically acceptable salt or ester thereof is administered in an amount ranging from about 1 mg/kg to about 10 mg/kg. In some embodiments, the sodium 2,2-dimethylbutanoate, or an equivalent dose of 2,2-dimethylbutyric acid or a CoA ester or carnitine ester thereof, or a pharmaceutically acceptable salt or ester thereof is administered in an amount ranging from about 1 mg/kg to about 15 mg/kg. In some embodiments, the sodium 2,2-dimethylbutanoate, or an equivalent dose of 2,2-dimethylbutyric acid or a CoA ester or carnitine ester thereof, or a pharmaceutically acceptable salt or ester thereof is administered in an amount ranging from about 1 mg/kg to about 30 mg/kg. In some embodiments, the sodium 2,2-dimethylbutanoate, or an equivalent dose of 2,2-dimethylbutyric acid or a CoA ester or carnitine ester thereof, or a pharmaceutically acceptable salt or ester thereof is administered at a concentration ranging from about 5 mg/kg to about 15 mg/kg. In some embodiments, administration is once a day (i.e., QD dosing). In some embodiments, administration is twice a day (i.e., BID dosing). In some embodiments, administration is three times a day (i.e., TID dosing).

In some embodiments, about 1 mg/kg to about 15 mg/kg of sodium 2,2-dimethylbutanoate, or an equivalent dose of 2,2-dimethylbutyric acid or a CoA ester or carnitine ester thereof, or a pharmaceutically acceptable salt or ester thereof is administered once daily (QD), e.g., about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, or about 15 mg/kg, including all ranges and values therebetween. In some embodiments, about 1 mg/kg of sodium 2,2-dimethylbutanoate, or an equivalent dose of 2,2-dimethylbutyric acid or a CoA ester or carnitine ester thereof, or a pharmaceutically acceptable salt, or ester thereof is administered once daily (QD). In some embodiments, about 3 mg/kg of sodium 2,2-dimethylbutanoate, or an equivalent dose of 2,2-dimethylbutyric acid or a CoA ester or carnitine ester thereof, or a pharmaceutically acceptable salt, or ester thereof is administered once daily (QD). In some embodiments, about 9 mg/kg of sodium 2,2-dimethylbutanoate, or an equivalent dose of 2,2-dimethylbutyric acid or a CoA ester or carnitine ester thereof, or a pharmaceutically acceptable salt, or ester thereof is administered once daily (QD). In some embodiments, about 10 mg/kg of sodium 2,2-dimethylbutanoate, or an equivalent dose of 2,2-dimethylbutyric acid or a CoA ester or carnitine ester thereof, or a pharmaceutically acceptable salt, or ester thereof is administered once daily (QD). In some embodiments, about 15 mg/kg of sodium 2,2-dimethylbutanoate, or an equivalent dose of 2,2-dimethylbutyric acid or a CoA ester or carnitine ester thereof, or a pharmaceutically acceptable salt, or ester thereof is administered once daily (QD).

In some embodiments, about 1 mg/kg to about 25 mg/kg of sodium 2,2-dimethylbutanoate, or an equivalent dose of 2,2-dimethylbutyric acid or a CoA ester or carnitine ester thereof, or a pharmaceutically acceptable salt, or ester thereof is administered two times a day (BID), e.g., about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, or about 25 mg/kg, including all ranges and values therebetween. In some embodiments, about 5 mg/kg to about 15 mg/kg of 2,2-dimethylbutyric acid, or an equivalent dose of a CoA ester or carnitine ester thereof, or a pharmaceutically acceptable salt or ester thereof is administered two times a day (BID). In some embodiments, about 3 mg/kg, about 5 mg/kg, about 9 mg/kg, about 10 mg/kg, or about 15 mg/kg of sodium 2,2-dimethylbutanoate, or an equivalent dose of 2,2-dimethylbutyric acid or a CoA ester or carnitine ester thereof, or a pharmaceutically acceptable salt or ester thereof is administered two times a day (BID). Thus, in some embodiments, the methods comprise administering a total daily dose of about 9 mg/kg, about 10 mg/kg, about 18 mg/kg, about 20 mg/kg, or about 30 mg/kg of sodium 2,2-dimethylbutanoate, or an equivalent dose of 2,2-dimethylbutyric acid or a CoA ester or carnitine ester thereof, or a pharmaceutically acceptable salt or ester thereof. In some embodiments, about 3 mg/kg of sodium 2,2-dimethylbutanoate, or an equivalent dose of 2,2-dimethylbutyric acid or a CoA ester or carnitine ester thereof, or a pharmaceutically acceptable salt or ester thereof is administered two times a day (BID). In such embodiments, the total daily dose administered is about 6 mg/kg of sodium 2,2-dimethylbutanoate, or an equivalent dose of 2,2-dimethylbutyric acid or a CoA ester or carnitine ester thereof, or a pharmaceutically acceptable salt or ester thereof. In some embodiments, about 5 mg/kg of sodium 2,2-dimethylbutanoate, or an equivalent dose of 2,2-dimethylbutyric acid or a CoA ester or carnitine ester thereof, or a pharmaceutically acceptable salt or ester thereof is administered two times a day (BID). In such embodiments, the total daily dose administered is about 10 mg/kg of sodium 2,2-dimethylbutanoate, or an equivalent dose of 2,2-dimethylbutyric acid or a CoA ester or carnitine ester thereof, or a pharmaceutically acceptable salt or ester thereof. In some embodiments, about 9 mg/kg of sodium 2,2-dimethylbutanoate, or an equivalent dose of 2,2-dimethylbutyric acid or a CoA ester or carnitine ester thereof, or a pharmaceutically acceptable salt or ester thereof is administered two times a day (BID). In some embodiments, about 10 mg/kg of sodium 2,2-dimethylbutanoate, or an equivalent dose of 2,2-dimethylbutyric acid or a CoA ester or carnitine ester thereof, or a pharmaceutically acceptable salt or ester thereof is administered two times a day (BID). In some embodiments, about 15 mg/kg of sodium 2,2-dimethylbutanoate, or an equivalent dose of 2,2-dimethylbutyric acid or a CoA ester or carnitine ester thereof, or a pharmaceutically acceptable salt or ester thereof is administered two times a day (BID). In such embodiments, the total daily dose administered is about 18 mg/kg of sodium 2,2-dimethylbutanoate, or an equivalent dose of 2,2-dimethylbutyric acid or a CoA ester or carnitine ester thereof, or a pharmaceutically acceptable salt or ester thereof. In some embodiments, about 10 mg/kg of sodium 2,2-dimethylbutanoate, or an equivalent dose of 2,2-dimethylbutyric acid or a CoA ester or carnitine ester thereof, or a pharmaceutically acceptable salt or ester thereof is administered two times a day (BID). In such embodiments, the total daily dose administered is about 20 mg/kg of sodium 2,2-dimethylbutanoate, or an equivalent dose of 2,2-dimethylbutyric acid or a CoA ester or carnitine ester thereof, or a pharmaceutically acceptable salt or ester thereof. In some embodiments, about 15 mg/kg of sodium 2,2-dimethylbutanoate, or an equivalent dose of 2,2-dimethylbutyric acid or a CoA ester or carnitine ester thereof, or a pharmaceutically acceptable salt or ester thereof is administered two times a day (BID). In such embodiments, the total daily dose administered is about 30 mg/kg of sodium 2,2-dimethylbutanoate, or an equivalent dose of 2,2-dimethylbutyric acid or a CoA ester or carnitine ester thereof, or a pharmaceutically acceptable salt or ester thereof. In some embodiments, the methods comprise administering about 3 mg/kg, about 9 mg/kg or about 15 mg/kg of sodium 2,2-dimethylbutanoate, or an equivalent dose of 2,2-dimethylbutyric acid or a CoA ester or carnitine ester thereof, or a pharmaceutically acceptable salt or ester thereof, twice a day (BID) for a total daily dose of about 6 mg/kg/day, about 18 mg/kg/day or about 30 mg/kg/day, respectively.

In some embodiments, the method comprises administering, once daily, about 0.84 mg/kg of 2,2-dimethylbutyric acid, or an equivalent dose of a pharmaceutically acceptable salt or ester thereof. In some embodiments, the method comprises administering, once daily, about 1 mg/kg of 2,2-dimethylbutyric acid, or an equivalent dose of a pharmaceutically acceptable salt or ester thereof. In some embodiments, the method comprises administering, once daily, about 2.5 mg/kg of 2,2-dimethylbutyric acid, or an equivalent dose of a pharmaceutically acceptable salt or ester thereof. In some embodiments, the method comprises administering, once daily, about 3 mg/kg of 2,2-dimethylbutyric acid, or an equivalent dose of a pharmaceutically acceptable salt or ester thereof. In some embodiments, the method comprises administering, twice daily, about 7.6 mg/kg of 2,2-dimethylbutyric acid, or an equivalent dose of a pharmaceutically acceptable salt or ester thereof. In some embodiments, the method comprises administering, once daily, about 8.4 mg/kg of 2,2-dimethylbutyric acid, or an equivalent dose of a pharmaceutically acceptable salt or ester thereof. In some embodiments, the method comprises administering, once daily, about 10 mg/kg of 2,2-dimethylbutyric acid, or an equivalent dose of a pharmaceutically acceptable salt or ester thereof. In some embodiments, the method comprises administering, twice daily, about 4.2 mg/kg of 2,2-dimethylbutyric acid, or an equivalent dose of a pharmaceutically acceptable salt or ester thereof. In some embodiments, the method comprises administering, twice daily, about 8.4 mg/kg of 2,2-dimethylbutyric acid, or an equivalent dose of a pharmaceutically acceptable salt or ester thereof. In some embodiments, the method comprises administering, twice daily, about 12.6 mg/kg of 2,2-dimethylbutyric acid, or an equivalent dose of a pharmaceutically acceptable salt or ester thereof.

### Pharmaceutical Compositions

In some embodiments, the present disclosure provides pharmaceutical compositions comprising one or more compounds, or pharmaceutically acceptable salts thereof.

In some embodiments, pharmaceutical compositions of the present disclosure are orally deliverable. The terms “orally deliverable” or “oral administration” herein include any form of delivery of a one or more compounds, pharmaceutically acceptable salts thereof, or a composition thereof to a subject wherein the one or more compounds, pharmaceutically acceptable salts thereof, or composition thereof is placed in the mouth of the subject, whether or not the agent or composition is swallowed. Thus “oral administration” includes buccal and sublingual as well as esophageal administration. In embodiments, the one or more compounds, pharmaceutically acceptable salts thereof, or composition thereof is placed in the mouth and swallowed.

In some embodiments, compositions of the present disclosure can be formulated as one or more dosage units. The terms “dose unit” and “dosage unit” herein refer to a portion of a pharmaceutical composition that contains an amount of a therapeutic agent suitable for a single administration to provide a therapeutic effect. Such dosage units may be administered one to a plurality (i.e., 1 to about 10, 1 to 8, 1 to 6, 1 to 4 or 1 to 2) of times per day, or as many times as needed to elicit a therapeutic response.

In some embodiments, a pharmaceutical composition provided herein further comprises a pharmaceutically acceptable carrier. In some embodiments, the pharmaceutically acceptable carrier enables the pharmaceutical compositions to be formulated as tablets, pills, dragees, capsules, gel capsules, liquids, gels, syrups, slurries, suspensions, and the like, for oral ingestion by a subject.

In some embodiments, suitable pharmaceutically acceptable carriers may be solid or liquid carriers. or liquid excipients. Such pharmaceutically acceptable liquid carriers can be aqueous or non-aqueous solutions, suspensions, and emulsions. Liquid carriers suitable for use in accordance with the present disclosure can be used in preparing solutions, suspensions, emulsions, syrups, elixirs, and pressurized compounds. The active ingredient (e.g., one or more compounds of the present disclosure) can be dissolved or suspended in a pharmaceutically acceptable liquid carrier such as water, an organic solvent, a mixture of both or pharmaceutically acceptable oils or fats. In some embodiments, the liquid carrier further comprises other suitable pharmaceutical additives such as solubilizers, emulsifiers, buffers, preservatives, sweeteners, flavoring agents, suspending agents, thickening agents, colors, viscosity regulators, stabilizers, or osmo-regulators.

In some embodiments, a pharmaceutical composition of the present disclosure comprises an aqueous carrier. Aqueous carriers suitable for use in accordance with the present disclosure include, but are not limited to, water, ethanol, alcoholic/aqueous solutions, glycerol, emulsions, or suspensions, including saline and buffered media.

Examples of non-aqueous solvents suitable for use in accordance with the present disclosure include, but are not limited to, propylene glycol, polyethylene glycol, vegetable oils such as olive oil, and injectable organic esters such as ethyl oleate.

Liquid pharmaceutical compositions may be prepared using compounds of the present disclosure, and any other solid excipients where the components are dissolved or suspended in a liquid carrier such as water, vegetable oil, alcohol, polyethylene glycol, propylene glycol, or glycerin.

Liquid pharmaceutical compositions may contain emulsifying agents to disperse uniformly throughout the composition and/or combination an active ingredient or other excipient that is not soluble in the liquid carrier. Emulsifying agents that may be useful in liquid compositions and/or combinations of the present disclosure include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions can also contain a viscosity enhancing agent to improve the mouth-feel of the product. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

A liquid composition can also contain a buffer such as gluconic acid, lactic acid, citric acid or acetic acid, sodium gluconate, sodium lactate, sodium citrate, or sodium acetate. Selection of excipients and the amounts used may be readily determined by the formulation scientist based upon experience and consideration of standard procedures and reference works in the field. In some embodiments, a pharmaceutical composition of the present disclosure comprises a solid carrier. Solid carriers suitable for use in accordance with the present disclosure, but are not limited to, inert substances such as lactose, starch, glucose, methyl-cellulose, magnesium stearate, dicalcium phosphate, mannitol, and the like.

In some embodiments, the solid compositions further comprise one or more substances acting as flavoring agents, lubricants, solubilizers, suspending agents, fillers, glidants, compression aids, binders, or tablet-disintegrating agents. In powders, the carrier can be a finely divided solid which is in admixture with the finely divided active compound. In tablets, the active compound is mixed with a carrier having the necessary compression properties in suitable proportions and compacted in the shape and size desired. The powders and tablets may contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins.

In some embodiments, a tablet may be made by compression or molding, optionally with one or more accessory ingredients. Compressed tablets may be prepared by compressing in a suitable machine the active ingredient in a free flowing form such as a powder or granules, optionally mixed with a binder, lubricant, inert diluent, preservative, disintegrant, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

In some embodiments, a pharmaceutical composition of the present disclosure comprises one or more diluents. Diluents increase the bulk of a solid pharmaceutical composition and/or combination, and may make a pharmaceutical dosage form containing the composition and/or combination easier for the patient and care giver to handle. Diluents for solid compositions and/or combinations include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT (r)), potassium chloride, powdered cellulose, sodium chloride, sorbitol, and talc.

In some embodiments, a pharmaceutical composition of the present disclosure comprises one or more disintegrants. The dissolution rate of a compacted solid pharmaceutical composition in the patient's stomach may be increased by the addition of a disintegrant to the composition and/or combination. Disintegrants include alginic acid, carboxymethylcellulose calcium, carboxymethylcellulose sodium (e.g., AC-DI-SOL and PRIMELLOSE), colloidal silicon dioxide, croscarmellose sodium, crospovidone (e.g., KOLLIDON and POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB), potato starch, and starch.

In some embodiments, a pharmaceutical composition of the present disclosure comprises one or more glidants. Glidants can be added to improve the flowability of a non-compacted solid composition and/or combination and to improve the accuracy of dosing. Excipients that may function as glidants include colloidal silicon dioxide, magnesium trisilicate, powdered cellulose, starch, talc, and tribasic calcium phosphate.

In some embodiments, a pharmaceutical composition of the present disclosure comprises one or more flavoring agents and/or flavor enhancers. Flavoring agents and flavor enhancers make the dosage form more palatable to the patient. Common flavoring agents and flavor enhancers for pharmaceutical products that may be included in the composition of the present disclosure include maltol, vanillin, ethyl vanillin, menthol, citric acid, fumaric acid, ethyl maltol, and tartaric acid.

In some embodiments, a pharmaceutical composition of the present disclosure comprises one or more sweetening agents. Sweetening agents such as aspartame, lactose, sorbitol, saccharin, sodium saccharin, sucrose, aspartame, fructose, mannitol, and invert sugar may be added to improve the taste.

In some embodiments, a pharmaceutical composition of the present disclosure comprises one or more of a preservative and/or chelating agents. Preservatives and chelating agents such as alcohol, sodium benzoate, butylated hydroxyl toluene, butylated hydroxyanisole, and ethylenediamine tetraacetic acid may be added at levels safe for ingestion to improve storage stability.

Solid pharmaceutical compositions that are compacted into a dosage form, such as a tablet, may include excipients whose functions include helping to bind the active ingredient and other excipients together after compression. Binders for solid pharmaceutical compositions and/or combinations include acacia, alginic acid, carbomer (e.g., carbopol), carboxymethylcellulose sodium, dextrin, ethyl cellulose, gelatin, guar gum, gum tragacanth, hydrogenated vegetable oil, hydroxyethyl cellulose, hydroxypropyl cellulose (e.g., KLUCEL), hydroxypropyl methyl cellulose (e.g., METHOCEL), liquid glucose, magnesium aluminum silicate, maltodextrin, methylcellulose, polymethacrylates, povidone (e.g., KOLLIDON, PLASDONE), pregelatinized starch, sodium alginate, and starch.

When a dosage form such as a tablet is made by the compaction of a powdered composition, the composition is subjected to pressure from a punch and dye. Some excipients and active ingredients tend to adhere to the surfaces of the punch and dye, which can cause the product to have pitting and other surface irregularities. A lubricant can be added to the composition and/or combination to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Solid and liquid compositions may also be dyed using any pharmaceutically acceptable colorant to improve their appearance and/or facilitate patient identification of the product and unit dosage level.

In some embodiments, the compounds of the disclosure are formulated in a composition disclosed in U.S. Pat. No. 8,242,172, in order to improve the physiological stability of the compound. Physiological stable compounds are compounds that do not break down or otherwise become ineffective upon introduction to a patient prior to having a desired effect. Compounds are structurally resistant to catabolism and thus, physiologically stable, or coupled by electrostatic or covalent bonds to specific reagents to increase physiological stability. Such reagents include amino acids such as arginine, glycine, alanine, asparagine, glutamine, histidine or lysine, nucleic acids including nucleosides or nucleotides, or substituents such as carbohydrates, saccharides and polysaccharides, lipids, fatty acids, proteins, or protein fragments. Useful coupling partners include, for example, glycol such as polyethylene glycol, glucose, glycerol, glycerin, and other related substances.

Physiological stability can be measured from a number of parameters such as the half-life of the compound or the half-life of active metabolic products derived from the compound. Certain compounds of the present disclosure have in vivo half-lives of greater than about fifteen minutes, preferably greater than about one hour, more preferably greater than about two hours, and even more preferably greater than about four hours, eight hours, twelve hours or longer. Although a compound is stable using this criteria, physiological stability cam also be measured by observing the duration of biological effects on the patient. Clinical symptoms which are important from the patient's perspective include a reduced frequency or duration, or elimination of the need for oxygen, inhaled medicines, or pulmonary therapy.

The concentration of a disclosed compound in a pharmaceutically acceptable mixture will vary depending on several factors, including the dosage of the compound to be administered, the pharmacokinetic characteristics of the compound(s) employed, and the route of administration. The agent may be administered in a single dose or in repeat doses. The dosage regimen utilizing the compounds of the present disclosure is selected in accordance with a variety of factors including type, species, age, weight, sex, and medical condition of the patient; the severity of the condition to be treated; the renal and hepatic function of the patient; and the particular compound or salt thereof employed. Treatments may be once administered daily or more frequently depending upon a number of factors, including the overall health of a patient, and the formulation and route of administration of the selected compound(s).

The compounds disclosed herein can be formulated in accordance with the routine procedures adapted for desired administration route. Accordingly, the compounds disclosed herein can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, PA.

In certain embodiments, a pharmaceutical composition of the present disclosure is prepared using known techniques, including, but not limited to mixing, dissolving, granulating, dragee-making, levigating, emulsifying, encapsulating, entrapping, or tableting processes.

### Methods of Treatment

In some embodiments, the sodium 2,2-dimethylbutanoate, or an equivalent dose of 2,2-dimethylbutyric acid or a CoA ester or carnitine ester thereof, or a pharmaceutically acceptable salt or ester thereof is administered to a subject in need thereof according to the methods disclosed herein is provided as single or divided (e.g., three times in a 24 hour period) doses, wherein the amount for each of the doses is determined by patient weight. According to a weight-based dosing regimen, each dose administered may be in a range of from about 0.5 mg/kg to about 30 mg/kg, e.g., about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12 mg/kg, about 15 mg/kg, about 20 mg/kg, about 25 mg/kg, about 30 mg/kg, inclusive of all values and subranges therebetween. In some embodiments, the dose is in a range of from about 1 mg/kg to about 30 mg/kg. In some embodiments, the dose ranges from about 1 mg/kg to about 10 mg/kg (e.g., about 1 mg/kg, about 3 mg/kg, or about 10 mg/kg) administered once daily. In some embodiments, the dose ranges from about 5 mg/kg to about 15 mg/kg (e.g., about 5 mg/kg, about 10 mg/kg, or about 15 mg/kg) administered twice daily). In some embodiments, the dose is in the range of from about 1 mg to about 100 g, e.g., about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1 g, about 2 g, about 3 g, about 4 g, about 5 g, about 6 g, about 7 g, about 8 g, about 9 g, about 10 g, about 15 g, about 20 g, about 25 g, about 30 g, about 35 g, about 40 g, about 45 mg, about 50 g, about 55 g, about 60 g, about 65 g, about 70 g, about 75 g, about 80 g, about 85 g, about 90 g, about 95 g, and about 100 g, inclusive of all values and subranges therebetween. Any of the above doses may a “therapeutically effective” amount as used herein.

In some embodiments, one or more compounds disclosed herein may be administered one or more times a day, e.g., 1, 2, 3, 4, 5, 6, 7, 8, 9, or 10 times a day. In some embodiments, one or more compounds disclosed herein may be administered one time a day (QD), two times a day (BID), or three times a day (TID). In some embodiments, one or more compounds disclosed herein may be administered two times a day (BID). In some embodiments, one or more compounds disclosed herein may administered to the patient for a period of time sufficient to efficacious for the treatment of an organic acidemia. In some embodiments, the treatment regimen is an acute regimen. In some embodiments, the treatment regimen is a chronic treatment regimen. In some embodiments, the patient is treated for 1 week, about 2 weeks, about 3 weeks, about 4 weeks, about 5 weeks, about 6 weeks, about 7 weeks, about 8 weeks, about 9, weeks about 10 weeks, about 20 weeks, about 30 weeks, about 40 weeks, about 50 weeks, about 60 weeks, about 70 weeks, about 80 weeks, about 90 weeks, about 100 weeks, about 1 year, about 2 years, about 3 years, about 4 years, about 5 years, about 6 years, about 7 years, about 8 years, about 9 years, about 10 years, about 15 years, about 20 years, about 30 years, about 40 years, about 50 years, about 60 years, about 70 years, about 80 years, or for the entirety of the patient's lifetime.

In some embodiments, the patient treated accordance with the methods provided herein is a newborn, or is about 1 month to 12 months old, about 1 year to 10 years old, about 10 to 20 years old, about 12 to 18 years old, about 20 to 30 years old, about 30 to 40 years old, about 40 to 50 years old, about 50 to 60 years old, about 60 to 70 years old, about 70 to 80 years old, about 80 to 90 years old, about 90 to 100 years old, or any age in between. In some embodiments, a patient treated in accordance with the methods disclosed herein is a newborn human. In some embodiments, the patient treated in accordance with the methods provided herein is between the age of newborn and 1 year old. In some embodiments, patient is between the age of 1 year old and 18 years old. In some embodiments, the patient is between the age of 1 year old and 5 years old. In some embodiments, the patient is between the age of 5 years old or 12 years old. In some embodiments, the patient is between the age of 12 years old and 18 years old. In some embodiments, the patient is at least 1 year old or older. In some embodiments, the patient is at least 2 years old or older. In some embodiments, the patient is between the ages of 2 years old and 5 years old, 2 years old and 10 years old, 2 years old and 12 years old, 2 years old and 15 years old, 2 years old and 18 years old, 5 years old and 10 years old, 5 years old and 12 years old, 5 years old and 15 years old or 5 years old and 18 years old.

In some embodiments, the patient is a pediatric (12 and under), an adolescent (13 to 17), an adult (18 to 65), or a geriatric (65 or older). In some embodiments, the pediatric patient is a newborn child, e.g., from 0 to 6 months. In some embodiments, the pediatric patient is an infant, aged 6 months to 1 year. In some embodiments, the pediatric patient is 6 months to 2 years old. In some embodiments, the pediatric patient is 2 years to 6 years old. In some embodiments, the pediatric patient is 6 years to 12 years old. In some embodiments, the child is under 10 years of age.

In some embodiments, the methods for treating the diseases provided herein improve or developmental or cognitive function in a subject. Such improvements in developmental or cognitive function may be as assessed by, e.g., the Bayley Scale of Infant Development, Wechsler Preschool and Primary Scale of Intelligence (WIPPSI), Wechsler Intelligence Scale for Children (WISC) or Wechsler Adult Intelligence Scale (WAIS). Some embodiments, an improvement in developmental or cognitive function may be assessed using the methods provided in the examples in US 2014/0343009, which is herein incorporated by reference in its entirety for all purposes.

In some embodiments, the methods provided herein improve control of muscle contractions by a patient as assessed by methods well known in the art, e.g., the Burke-Fahn-Marsden rating scale. In certain aspects, the methods provided herein decrease the occurrence of metabolic decompensation episodes, characterized by, e.g., vomiting, hypotonia, and alteration in consciousness.

In some embodiments, the methods provided herein are suitable in patients that have received a liver transplant (e.g., OLT) or a kidney transplant or a liver and kidney transplant.

In some embodiments, the methods provided herein improve renal function. In certain embodiments, the methods provided herein decrease the need for kidney transplant, liver transplant or both.

In some embodiments, the methods provided herein decrease the requirement for hospitalization. In certain embodiments, the methods provided herein decrease the length and/or frequency of hospitalization.

In some embodiments, such methods reduce the production of toxic metabolites in a subject. Advantageously and surprisingly, the compounds and methods of the present disclosure are able to reduce the production of toxic metabolites in various tissue throughout the body in order to achieve disease remediation. In some embodiments, the metabolites are metabolites produced in the liver. In some embodiments, the metabolites are metabolites produced in the muscle. In some embodiments, the metabolites are metabolites produced in the brain. In some embodiments, the metabolites are metabolites produced in the kidney. In some embodiments, the metabolites are metabolites produced in any organ tissue. In some embodiments, the metabolite is a metabolite of one or more of a branched chain amino acid, methionine, threonine, odd-chain fatty acids and cholesterol. In some embodiments, the metabolites can be propionyl-CoA. In some embodiments, the metabolite is methylmalonyl-CoA. In some embodiments, the metabolite is 2-methylcitric acid (MCA). In some embodiments, the metabolite is propionyl-carnitine. In some embodiments, the metabolite is 3-OH propionate.

In some embodiments, the methods of the disclosure increase clearance (e.g., excretion or elimination) of one or more toxic metabolites disclosed herein. Non-limiting examples of such toxic metabolites that may experience an increase in clearance include methylmalonyl-CoA, propionyl-CoA, or a combination thereof. In embodiments in which clearance of toxic metabolites is increased, the levels of such metabolites excreted and/or eliminated may be increased compared to the levels of the toxic metabolites excreted and/or eliminated at baseline. In embodiments, metabolite levels may be measured in urine, bile, sweat, saliva, tears, milk, or stool. In embodiments, increased clearance may occur throughout treatment or intermittently during treatment according to the methods disclosed herein. Without being bound by theory, increased clearance of a toxic metabolite may also cause increases in related metabolites. For example, when a patient experiences an increase in clearance of propionyl-CoA, the patient may also exhibit a concomitant rise in the levels of propionyl-carnitine (C3), propionic acid, propionate, propionyl-carnitine and propionyl-glycine, or other propionyl derivative. Non-limiting examples propionyl and methylmalonyl derivatives that may be increased compared to baseline as a result of increased clearance of methylmalonyl-CoA and/or propionyl-CoA include methylmalonic acid, methylmalonyl-carnitine, propionic acid, propionate, propionyl-carnitine, propionyl-glycine, and the like

In some embodiments, the methods of the disclosure reduce levels of at least one metabolite of a branched chain amino acid is (e.g., propionyl-CoA and/or methylmalonyl-CoA levels) by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, or by any values in between as compared to baseline or control. In some embodiments, the level can be reduced by at least 87.5% as compared to baseline or control. In some embodiments, at least one metabolite of a branched chain amino acid (e.g., propionyl-CoA and/or methylmalonyl-CoA levels) is reduced by an amount ranging from about 1% to about 100%, e.g., about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween. In some embodiments, the metabolite is a metabolite of one or more of a branched chain amino acid, methionine, threonine, odd-chain fatty acids and cholesterol. In some embodiments, the metabolite (or metabolites), such as propionyl-CoA and/or methylmalonyl-CoA, are reduced to a level that achieves the therapeutic effects in treating organic acidemia. In some embodiments, the metabolite is propionyl-CoA and/or methylmalonyl-CoA. In some embodiments, the metabolite is 3-hydroxypropionic acid, methylcitrate, methylmalonic acid, propionyl-glycine, or propionyl-carnitine, or combinations thereof. In some embodiments, metabolite is 2-ketoisocaproate, isovaleryl-CoA, 3-methylcrotonyl-CoA, 3-methylglutaconyl-CoA, 3-OH-3-methylglutaryl-CoA, 2-keto-3-methylvalerate, 2-methylbutyryl-CoA, tiglyl-CoA, 2-methyl-3-OH-butyryl-CoA, 2-methyl-acetoacetyl-CoA, 2-ketoisovalerate, isobutyryl-CoA, methylacrylyl-CoA, 3-OH-isobutyryl-CoA, 3-OH-isobutyrate, methylmalonic semialdehyde, propionyl-CoA, or methylmalonyl-CoA, or combinations thereof.

In some embodiments, the methods of the present disclosure reduce MCA levels by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, or by any values in between as compared to baseline and/or control. In some embodiments, the MCA levels are reduced by an amount ranging from about 5% to about 100%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween as compared to baseline and/or control.

In some embodiments, the methods of the present disclosure reduce a plasma MCA: citric acid (MCA: CA) ratio by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, or by any values in between as compared to baseline and/or control. In some embodiments, the MCA: CA ratio is reduced by an amount ranging from about 5% to about 100%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween as compared to baseline and/or control.

In some embodiments, the methods of the present disclosure reduce propionyl-carnitine levels by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, or by any values in between as compared to baseline and/or control. In some embodiments, the propionyl-carnitine levels are reduced by an amount ranging from about 5% to about 100%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween as compared to baseline and/or control.

In some embodiments, the methods of the present disclosure reduce a plasma propionyl-carnitine to acetyl carnitine ratio by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, or by any values in between as compared to baseline and/or control. In some embodiments, the plasma propionyl-carnitine to acetyl carnitine ratio is reduced by an amount ranging from about 5% to about 100%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween as compared to baseline and/or control.

In some embodiments, the methods of the present disclosure reduce 3-OH propionate levels by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, or by any values in between as compared to baseline and/or control. In some embodiments, the 3-OH propionate levels are reduced by an amount ranging from about 5% to about 100%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween as compared to baseline and/or control.

In some embodiments, the methods of the present disclosure reduce ammonia (NH₃) levels by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, or by any values in between as compared to baseline and/or control. In some embodiments, the NH₃ levels are reduced by an amount ranging from about 5% to about 100%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween as compared to baseline and/or control.

In some embodiments, the methods of the present disclosure reduce an anion gap by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, or by any values in between as compared to baseline and/or control. In some embodiments, the anion gap is reduced by an amount ranging from about 5% to about 100%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween as compared to baseline and/or control.

In some embodiments, the methods of the present disclosure reduce carnitine levels (e.g., total, free and/or esterified carnitine levels), by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, or by any values in between as compared to baseline and/or control. In some embodiments, the carnitine levels (e.g., total, free and/or esterified carnitine levels), are reduced by an amount ranging from about 5% to about 100%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween as compared to baseline and/or control.

In some embodiments, the methods of the present disclosure reduce ketone levels (e.g., β-OH butyrate) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, or by any values in between as compared to baseline and/or control. In some embodiments, ketone (e.g., β-OH butyrate) levels are reduced by an amount ranging from about 5% to about 100%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween as compared to baseline and/or control.

In some embodiments, the methods of the present disclosure reduce lactic acid levels by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, or by any values in between as compared to baseline and/or control. In some embodiments, lactic acid levels are reduced by an amount ranging from about 5% to about 100%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween as compared to baseline and/or control.

In some embodiments, the methods of the present disclosure reduce ketone urine levels by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, or by any values in between as compared to baseline and/or control. In some embodiments, ketone urine levels are reduced by an amount ranging from about 5% to about 100%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween as compared to baseline and/or control.

In some embodiments, the methods of the present disclosure reduce organic acid levels by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, or by any values in between as compared to baseline and/or control. In some embodiments, organic acid levels are reduced by an amount ranging from about 5% to about 100%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween as compared to baseline and/or control.

In some embodiments, the methods of the present disclosure reduce acylglycines levels by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, or by any values in between as compared to baseline and/or control. In some embodiments, acylglycines levels are reduced by an amount ranging from about 5% to about 100%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween as compared to baseline and/or control.

In some embodiments, the methods of the present disclosure reduce the frequency of metabolic decompensation events requiring an emergency room visit or hospitalization by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, or by any values in between as compared to baseline and/or control. In some embodiments, the frequency of metabolic decompensation events requiring an emergency room visit or hospitalization are reduced by an amount ranging from about 5% to about 100%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween as compared to baseline and/or control. In some embodiments, the frequency of metabolic decompensation events requiring an emergency room visit or hospitalization is reduced overall and per dose level interval.

In some embodiments, the methods of the present disclosure reduce the frequency of days in the hospital required for treatment/resolution of metabolic decompensations by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, or by any values in between as compared to baseline and/or control. In some embodiments, the methods of the present disclosure reduce the number of days in the hospital required for treatment/resolution of metabolic decompensations while the patient is being treated according to the disclosure by at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least 20 days or more, as compared to baseline and/or control. In some embodiments, the frequency of days in the hospital required for treatment/resolution of metabolic decompensations is reduced by an amount ranging from about 5% to about 100%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween as compared to baseline and/or control. In some embodiments, the frequency of days in the hospital required for treatment/resolution of metabolic decompensations is reduced overall and per dose level interval.

In some embodiments, the methods of the present disclosure reduce the frequency of episodes and/or days requiring use of a home emergency treatment protocol for metabolic decompensations by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, or by any values in between as compared to baseline and/or control. In some embodiments, the methods of the present disclosure reduce the number of days requiring use of a home emergency treatment protocol for metabolic decompensations while the patient is being treated according to the disclosure by at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least 20 days or more or more as compared to baseline and/or control. In some embodiments, the frequency of episodes and/or days requiring use of a home emergency treatment protocol for metabolic decompensations are reduced by an amount ranging from about 5% to about 100%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween as compared to baseline and/or control. In some embodiments, the frequency of episodes and/or days requiring use of a home emergency treatment protocol for metabolic decompensations are reduced overall and per dose level interval.

In some embodiments, the methods of the present disclosure reduce QT corrected (QTc) intervals by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, or by any values in between as compared to baseline and/or control. In some embodiments, the QTc intervals are reduced by an amount ranging from about 5% to about 100%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween as compared to baseline and/or control.

In some embodiments, the methods of the present disclosure comprise improving cardiac disease as evaluated by a reduction in the left ventricular ejection fraction. In some embodiments, the left ventricular ejection fraction is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, or by any values in between as compared to baseline and/or control. In some embodiments, the left ventricular ejection fraction is reduced by an amount ranging from about 5% to about 100%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween as compared to baseline and/or control.

In some embodiments, the methods of the present disclosure reduce plasma and urinary metabolites by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, or by any values in between as compared to baseline and/or control. In some embodiments, the plasma and urinary metabolites are reduced by an amount ranging from about 5% to about 100%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween as compared to baseline and/or control. In some embodiments, the plasma and urinary metabolite is acylcarnitine, acylglycine, and acylglucuronide metabolites, or combinations thereof.

In some embodiments, the methods of the present disclosure reduce plasma fibroblast growth factor 21 (FGF-21) levels by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, or by any values in between as compared to baseline and/or control. In some embodiments, the plasma FGF-21 levels are reduced by an amount ranging from about 5% to about 100%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween as compared to baseline and/or control.

In some embodiments, the methods of the present disclosure comprise increasing plasma sodium 2,2-dimethylbutyrate levels by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, or by any values in between as compared to baseline and/or control. In some embodiments, the plasma sodium 2,2-dimethylbutyrate levels are increased by an amount ranging from about 5% to about 100%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween as compared to baseline and/or control.

In some embodiments, the methods of the present disclosure comprise improving the quality of life of the patient during the administration period, as compared to the quality of life of the patient prior to the administration patient. Various assessment tools have been developed to evaluate a patient's health-related quality of life (HrQOL) such as the MetabQOL 1.0, the PedsQL questionnaire and, the Clinician-Reported Global Assessments of Severity and Change questionnaire.

In some embodiments, the methods of the present disclosure comprise improving the quality of life of a patient as evaluated by a MetabQOL 1.0 score. A MetabQL 1.0 questionnaire is a 28-item check list designed to assess quality of life in patients with organic acidemias, urea cycle disorders and maple syrup urine disease for patients between the ages of 8-18 years. (Zeltner et al. JIMD Rep. 2017; 37:27-35). The test is administered verbally and takes approximately 10 minutes. Specifically, the questionnaire consists of fifty questions answered using 5-point Likert frequency scales (options: never=0, seldom=1, sometimes=2, often=3, always=4), with an additional answer option (e.g., “no problem with this”) for questions not applicable for all patients. Two questions assess disease severity during the last 12 months: (1) the disease has been not bad at all=0, slightly bad=1, medium bad=2, bad=3, or very bad=4 and (2) number of hospital admissions never=0, once=1, twice=2, three to five times=3, or six times or more=4. Item scores can be aggregated to scale scores, which represent the core dimensions of physical, mental, and social HrQol and a HrQol total score. In some embodiments, the methods of the present disclosure comprise decreasing the patient's MetabQOL 1.0 scores during and/or after the administration period as compared to the score prior to the administration period. The decrease, in one embodiment, is by from 4 to 0, from 4 to 1, from 4 to 2, from 4 to 3, from 3 to 0, from 3 to 1, from 3 to 2, from 2 to 0, from 2 to 1, or from 1 to 0. In some embodiments, the MetabQoL 1.0 decreases by 1, 2, 3, or 4.

In some embodiments, the methods of the present disclosure comprise improving the quality of life of a patient as evaluated by a PedsQL questionnaire. The PedsQL questionnaire measures parent self-reported physical, emotional, social, and cognitive function, as well as communication and worry. Thirty-six items are reported on a 5-point Likert frequency scale, similar to the scoring for MetabQOL 1.0. (Zeltner et al. JIMD Rep. 2017; 37:27-35). The test is administered verbally and takes approximately 10 minutes. The Parent HRQOL Summary Score (20 items) is computed as the sum of the items divided by the number of items answered in the Physical, Emotional, Social, and Cognitive Functioning Scales. The Family Functioning Summary Score (8 items) is computed as the sum of the items divided by the number of items answered in the Daily Activities and Family Relationships Scales. (Varni et al, Med Care 1999; 37 (2): 126-139; Splinter et al, J. Genet Counsel 2016; 25:936-944). In some embodiments, the methods of the present disclosure comprise decreasing the patient's PedsQL scores during and/or after the administration period as compared to the score prior to the administration period. The decrease, in one embodiment, is by from 4 to 0, from 4 to 1, from 4 to 2, from 4 to 3, from 3 to 0, from 3 to 1, from 3 to 2, from 2 to 0, from 2 to 1, or from 1 to 0. In some embodiments the patient's PedsQL score decreases by 1, 2, 3, 4, or 5.

In some embodiments, the methods of the present disclosure comprise improving the quality of life of a patient as evaluated by a Clinician-Reported Global Assessments of Severity and Change score. Clinician-Reported Global Assessments of Severity and Change questionnaire is a 7-point scale that permits a clinician to rate the severity of the patient's illness at the time of assessment, relative to the clinicians' past experiments with patients who have the same diagnosis. The clinician asks, “considering the total clinical experience with this particular population, how ill is the patient at this time?” and then provides the following ratings: normal, not at all ill=1; borderline mentally ill=2; mildly ill=3; moderately ill=4; markedly ill=5; severely ill=6; and among the most extremely ill patients=7. In some embodiments, the methods of the present disclosure comprise decreasing the patient's Clinician-Reported Global Assessments of Severity and Change scores during and/or after the administration period as compared to the score prior to the administration period. The decrease, in one embodiment, is by from 7 to 1, from 7 to 2, from 7 to 3, from 7 to 4, from 7 to 5, from 7 to 6, from 6 to 1, from 6 to 2, from 6 to 3, from 6 to 4, from 6 to 5, from 5 to 1, from 5 to 2, from 5 to 3, from 5 to 4, from 4 to 1, from 4 to 2, from 4 to 3, from 3 to 1, from 3 to 2, or from 2 to 1. In some embodiments, the patient's Clinician-Reported Global Assessments of Severity and Change score decreases by 1, 2, 3, 4, 5, or 7.

The methods of the present disclosure can be combined with other therapies used in the treatment of metabolic diseases (including organic acidemias, e.g., PA or MMA) which can be administering subsequently, simultaneously, or sequentially (e.g., before or after) with the compounds of the disclosure (e.g., 2,2-dimethylbutyric acid, or CoA esters or carnitine esters thereof, or pharmaceutically acceptable salts, solvates, or esters thereof). Non-limiting examples of additional therapeutic agent which can be combined with the methods disclosed herein include: L-carnitine; glucose; L-arginine; Polycal (maltodextrin-based carbohydrate supplement); ammonia scavengers used to treat acute hyperammonemia, such as N-carbamyl-glutamate, sodium benzoate, sodium phenyl acetate, sodium phenylbutyrate, glycerol phenylbutyrate; antibiotics used to reduce the intestinal flora, such as metronidazole, amoxicillin or cotrimoxazole; vitamin B12 (in B12-responsive MMA patients); biotin; growth hormone therapy; low-protein diets; antioxidant therapies, such as N-acetylcysteine, cysteamine or a-tocotrienol quinone; and anaplerotic therapies, such as citrate, glutamine, ornithine a-ketoglutarate or pro-drugs of succinate; and essential amino acids such as norvaline, methionine, isoleucine, or threonine. In some embodiments, the additional therapeutic agent which can be combined with the methods disclosed herein is a messenger RNA therapeutic. In some embodiments, the messenger RNA therapeutic is mRNA-3927 or mRNA-3704. mRNA-3927 includes two mRNAs that encode for the alpha and beta subunits of the mitochondrial enzyme propionyl-CoA carboxylase (PCC), encapsulated within a lipid nanoparticle (LNP) and can be used to restore missing or dysfunctional proteins that cause PA. mRNA-3704 consists of mRNA encoding human MUT, the mitochondrial enzyme commonly deficient in MMA, encapsulated within a LNP. It is contemplated that the compounds of the present disclosure can be combined with mRNA-3927 or mRNA-3704 therapy, because the compounds of the present disclosure will reduce the levels of toxic metabolites disclosed herein, whereas mRNA-3927 or mRNA-3704 is target primarily the liver. In some embodiments, the compounds of the present disclosure may be used in patient with an organic acidemia after said patients receives a liver transplant. In some embodiments, the compounds of the disclosure are administered in combination with an AAV therapy, such as the AAV therapy from LogicBio (LB-001).

### EXAMPLES

### The Following Examples are Offered by Way of Illustration and not by Way of Limitation.

List of Abbreviations and Definitions of Terms:

| Abbreviation | Definition |
|---|---|
| ACDH | acyl-CoA dehydrogenase |
| AUC | area under the concentration time curve |
| C2 | acetyl-carnitine |
| C3 | propionyl-carnitine |
| CL | clearance rate |
| C4 | methylmalonyl-carnitine |
| Cₘₐₓ | maximum concentration |
| CNS | central nervous system |
| CoASH | coenzyme A |
| CPS1 | carbamoyl phosphate synthetase I |
| CS | citrate synthase |
| DMBA | 2,2-dimethylbutanoic acid |
| GLP | Good Laboratory Practice |
| EC₅₀ | effective concentration, 50% |
| EC₉₀ | effective concentration, 90% |
| Compound 5-CoA | 2,2-dimethylbutyryl-coenzyme A |
| HT-MS/MS | high-throughput mass spectrometry |
| IRB | Institutional Review Board |
| MRM | multi-reactant monitoring |
| MSMS | tandem mass spectrometry |
| LC-MS/MS | liquid chromatography mass spectrometry |
| M-acid | methylmalonic acid |
| MCA | 2-methylcitric acid |
| M-CoA | methylmalonyl-CoA |
| MMA | methylmalonic acidemia |
| MUT | methylmalonyl-CoA mutase |
| NAGS | N-acetylglutamate synthase |
| OMIM | Online Mendelian Inheritance in Man |
| PA | propionic acidemia |
| PCC | propionyl-CoA carboxylase |
| PCCA | α subunit of propionyl-CoA carboxylase |
| PCCB | β subunit of propionyl-CoA carboxylase |
| P-CoA | propionyl coenzyme A |
| PDH | pyruvate dehydrogenase |
| pHeps | primary hepatocytes |
| PK | pharmacokinetics |
| SD | standard deviation |
| SIL | Stable isotope labeling |
| TCA | tricarboxylic acid |

### Example 1

Reduction of Propionyl-CoA Levels from all Sources Following Compound 5 Treatment in PA Primary Hepatocytes

Primary hepatocytes isolated from livers of propionic acidemia patients are treated with increasing doses of Compound 5 (0, 0.1, 0.3, 1, 3, 10, 30, 100 μM) for 30 minutes. After 30 minutes the cells are challenged with the different sources of P-CoA, which may include 13C-KIVA (1 mM), 13C-isoleucine (3 mM), 13C-threonine (5 mM), 13C-methionine (5 mM), 13C-heptanoate (100 μM), or 13C-propionate (5 mM) for 60 minutes. At the end of the challenge period, media is removed and the cells are lysed with 70% MeCN and 0.1% TFA containing 100 μM of ethymalonyl-CoA as an internal standard and harvested. Cell lysates are processed for HTMS/MS analysis.

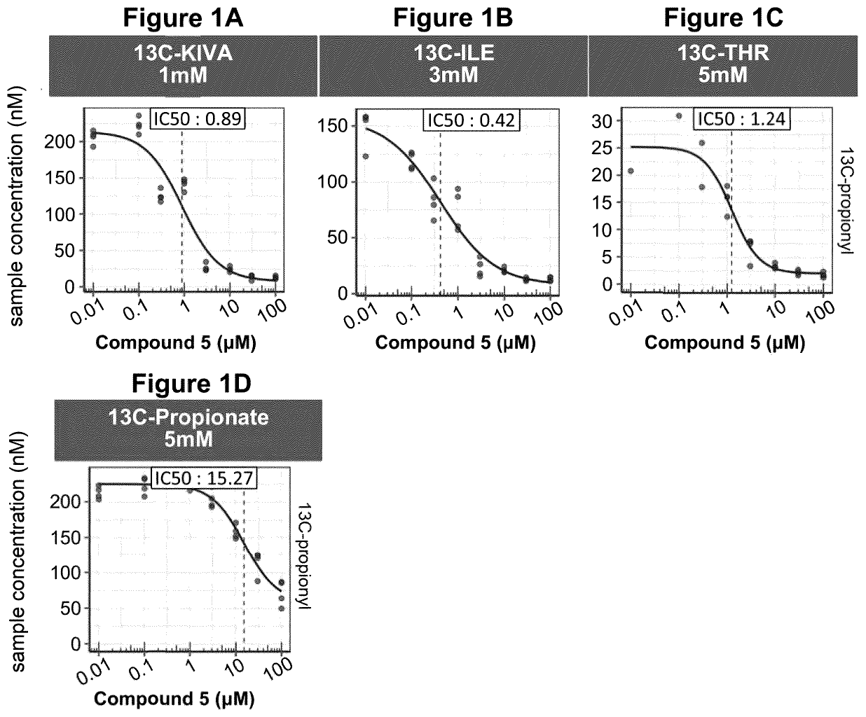

Treatment of primary hepatocytes isolated from livers of propionic acidemia patients with Compound 5 resulted in a dose-dependent reduction of intracellular propionyl-CoA from all sources investigated (FIG. 1A-1D). This indicates that treatment with Compound 5 alleviates the primarily metabolic defect (accumulation of propionyl-CoA) in primary hepatocytes of propionic acidemia patients.

### Example 2

Reduction of Methylmalonyl-CoA and Propionyl-CoA Levels from all Sources Following Compound 5 Treatment of MMA Primary Hepatocytes

Primary hepatocytes isolated from livers of methylmalonic acidemia patients are treated with increasing doses of Compound 5 (0, 0.1, 0.3, 1, 3, 10, 30, 100 M) for 30 min. After 30 minutes the cells are challenged with the different sources of P-CoA, which may include 13C-KIVA (1 mM), ¹³C-isoleucine (3 mM), ¹³C-threonine (5 mM), ¹³C-methionine (5 mM), ¹³C-heptanoate (100 μM), or ¹³C-propionate (5 mM) for 60 minutes. At the end of the challenge period, media is removed and the cells are lysed with 70% MeCN and 0.1% TFA containing 100 μM of ethymalonyl-CoA as an internal standard and harvested. Cell lysates are processed for HTMS/MS analysis.

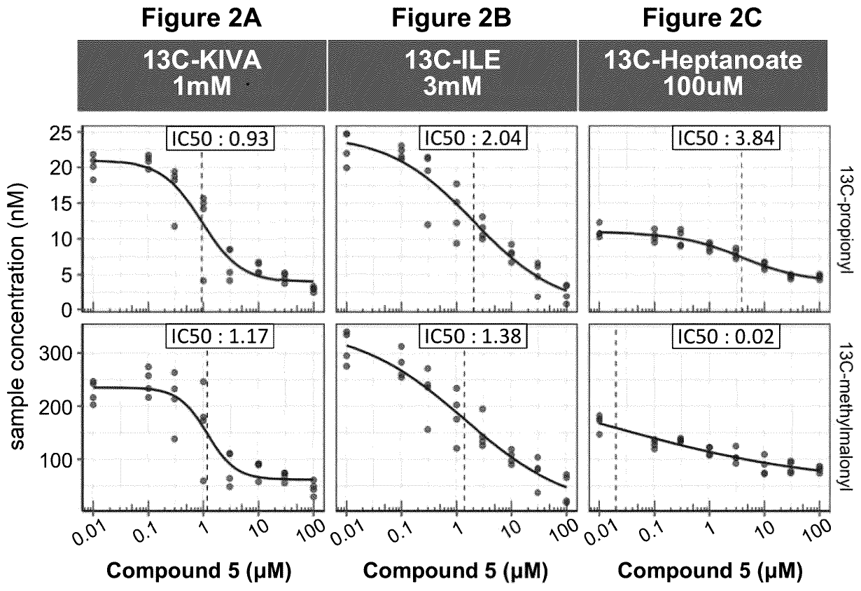

Treatment of primary hepatocytes isolated from livers of methylmalonic academia patients with Compound 5 resulted in a dose-dependent reduction of intracellular propionyl-CoA and methylmalonyl-CoA from all sources investigated (FIG. 2A-2C). This indicates that treatment with Compound 5 alleviates the primarily metabolic defect (accumulation of propionyl-CoA and methylmalonyl-CoA) in primary hepatocytes of methylmalonic acidemia patients.

### Example 3

### Effect on Ureagenesis Following Compound 5 Treatment

For this experiment, we deployed the HemoShear REVEAL-Tx™ technology, which is based on the cone-and-plate configuration or viscometer combined with a porous polycarbonate membrane that mimics a filtering layer of sinusoidal endothelial cells (see: Dash A, Deering TG, Marukian S, et al. Physiological Hemodynamics and Transport Restore Insulin and Glucagon Responses in a Normal Glucose Milieu in Hepatocytes In Vitro. 73rd Scientific Sessions of the American Diabetes Association, 2013 (Chicago), and U.S. Pat. Nos. 7,811,782 and 9,500,642, and 9,617,521, each of which is incorporated by reference herein in its entirety).

Hepatocytes from propionic academia patients are plated in a collagen gel sandwich on one side of the membrane replicating the polarized orientation found in vivo within the hepatic sinusoids. On the other side, medium is continuously perfused and surface shear rates are applied across a range of physiological values derived from sinusoidal flow rates in vivo while also controlling transport in the system with in- and out-flow tubing to each compartment. Effectively, this creates a flow-based culture system where hepatocytes are shielded from direct effects of flow, as they would be in vivo, but perfusion, nutrient gradients, and interstitial fluid movement are maintained. Under these conditions, human primary hepatocytes in the technology restore in vivo-like morphology, metabolism, transport, and CYP450 activity, and do not de-differentiate.

Hepatocytes are treated with increasing doses of Compound 5 (0, 0.1, 0.3, 1, 3, 10, 30, 100 μM) in the HemoShear REVEAL-Tx™ technology from day 5 to day 7. At day 7, islands of cells grown on membrane are cut and placed in 12-well plates and cultured under the same treatment conditions. ¹⁵N—NH₄Cl is added to each well and cells are incubated for 4 hrs. After 4 hrs, cells are washed 2× in saline solution and lysed, scraped and harvested using 80% methanol. ¹⁵N-urea is measured by GCMSMS.

Treatment of primary hepatocytes isolated from livers of propionic acidemia patients with Compound 5 resulted in a dose-dependent increase in ¹⁵N-urea. This result indicates that treatment with Compound 5 has an effect in improving ureagenesis.

### Example 4

### Reduction of Isovaleryl-CoA in a Primary Hepatocyte Model of Isovaleric Acidemia Following Compound 5 Treatment

Primary hepatocytes are treated with increasing doses of Compound 5 (0, 0.1, 0.3, 1, 3, 10, 30, 100 μM) with and without an inhibitor for isovaleryl-CoA dehydrogenase for 30 min. After 30 min the cells are challenged with 13C-leucine. At the end of the challenge period, media is removed and the cells are lysed with 70% MeCN and 0.1% TFA containing 100 μM of ethymalonyl-CoA as an internal standard and harvested. Cell lysates are processed for HTMS/MS analysis.

Treatment of primary hepatocytes with Compound 5 resulted in a dose-dependent reduction of intracellular isovaleryl-CoA derived from ¹³C-leucine. This indicates that treatment with Compound 5 alleviates the primarily metabolic defect (accumulation of isovaleryl-CoA) in a primary hepatocyte model of isovaleric academia.

### Example 5

Pharmacologic Activity of Compound 5 in Primary Hepatocytes from PA and MMA Patients

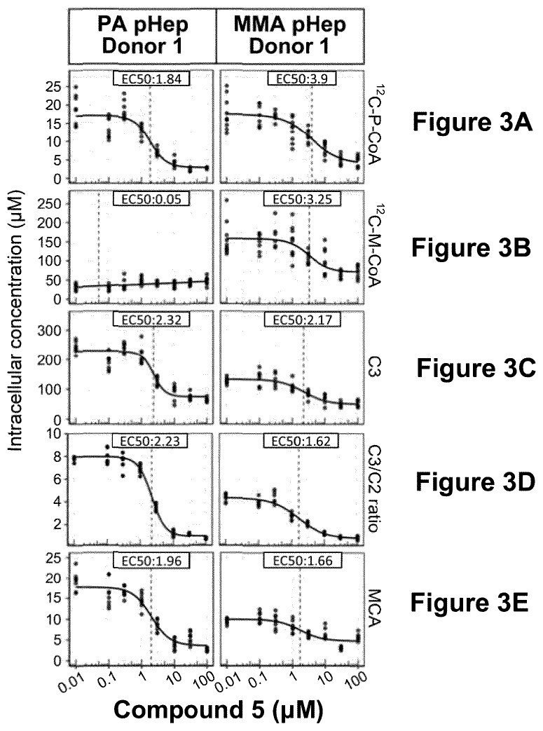

Representative activity data for Compound 5 in primary hepatocytes (pHeps) from PA and MMA donors was demonstrated using the HemoShear REVEAL-Tx™ Technology (FIGS. 3A-3E). Biomarker levels are normalized to cell counts and cell volume to account for differences in the number of cells plated for each donor. As shown in FIG. 3A, Compound 5 dose-dependently reduced P-CoA in PA and MMA pHeps with EC₅₀ values of 1.84 μM and 3.90 M, respectively. Compound 5 reduced M-CoA in the MMA pHeps with an EC₅₀ value of 3.25 μM (FIG. 3B). Analysis of PA pHep cell lysates demonstrated an apparent background level of ¹²C-M-CoA of ˜25-50 μM when measured by LC-MS/MS. This is most likely due to the presence of ¹²C-succinyl-CoA in the sample, which has the same mass. In experiments described below, M-CoA is labeled with ¹³C to determine a more accurate percent reduction. Compound 5 reduced the C3 concentration and the C3/C2 ratio with an EC₅₀ similar to that for the reduction in P-CoA (FIGS. 3C, 3D). MCA is dramatically reduced in both the PA and MMA donors, with an EC₅₀ of 1.96 μM and 1.66 μM, respectively (FIG. 3E).

The summary data for all 3 PA and 3 MMA donors is presented in Table 2. The EC₉₀ values for P-CoA reduction were 18.4±11.3 μM and 36.1±30.1 μM, in PA and MMA pHeps, respectively. Similarly, Compound 5 reduced the concentration of C3 in PA and MMA pHeps with EC₉₀ values of 30.8±26.4 μM and 18.1±16.2 μM, respectively. The EC₉₀ value for reduction in MCA in PA (7.9±3.6 μM) and MMA (7.5±6.4 μM) pHeps was lower than for the other biomarkers. The average EC₉₀ value across all biomarkers was 17.1±13.4 μM, and 30 μM was selected as a fixed concentration to determine the reduction across each biomarker to allow a uniform comparison. The average reduction in P-CoA levels in PA and MMA pHeps at 30 μM was −78.8±10.9% and −74.2±11.6% and for C3 level reductions were −68.9±14.6% and −65.9±10.7%, respectively. The average reduction (expressed as log 2 fold change) in the C3/C2 ratio was −2.1±1.2 in PA pHeps and −2.2±0.2 in MMA pHeps. MCA was reduced by −78.6±12.9% in PA pHeps and −66.7±14.9% in MMA pHeps. Overall, the EC₉₀ values for reduction in biomarker concentrations were consistent across all the biomarkers, suggesting that Compound 5 has a “global” effect on correcting relevant metabolic abnormalities in PA and MMA consistent with the biochemical pathways driving these disease phenotypes and thus supports its therapeutic potential in both disorders (Table 2).

TABLE 2
PA and MMA Biomarker Levels and EC₅₀ and EC₉₀ Values
of Compound 5 in the HemoShear Technology

| | Disease | % change | Curve | | |
|---|---|---|---|---|---|
| Biomarker | pHeps | at 30 μM | count | EC₅₀ μM | EC₉₀ μM |
| ¹²C-Propionyl- | PA | −78.8 ± 10.9 | 6 | 1.9 ± 1 | 18.4 ± 11.3 |
| CoA (P-CoA) | MMA | −74.2 ± 11.6 | 5 | 2.1 ± 1.5 | 36.1 ± 30.1 |
| ¹²C-Methylmalonyl- | PA | NA | NA | NA | NA |
| CoA (M-CoA) | MMA | −55 ± 6.6 | 3 | 2 ± 1.2 | 12.6 ± 12.9 |
| ¹²C-Propionyl- | PA | −69.4 ± 16.2 | 5 | 2.6 ± 1.9 | 32.1 ± 29.3 |
| Carnitine (C3) | MMA | −65.9 ± 10.7 | 4 | 1.4 ± 0.5 | 18.1 ± 16.2 |
| ¹²C-Acetyl- | PA | 54 ± 80.6 | 3 | 4.8 ± 0.2 | 20.7 ± 13.1 |
| Carnitine (C2) | MMA | 44.6 ± 26.8 | 3 | 4.3 ± 2.2 | 31.3 ± 26.9 |
| 2-Methylcitric acid | PA | −78.6 ± 12.9 | 5 | 1.1 ± 0.5 | 7.9 ± 3.6 |
| (2MC) | MMA | −66.7 ± 14.9 | 5 | 0.8 ± 0.5 | 7.5 ± 6.4 |

Values are average ± standard deviation
NA—Not applicable

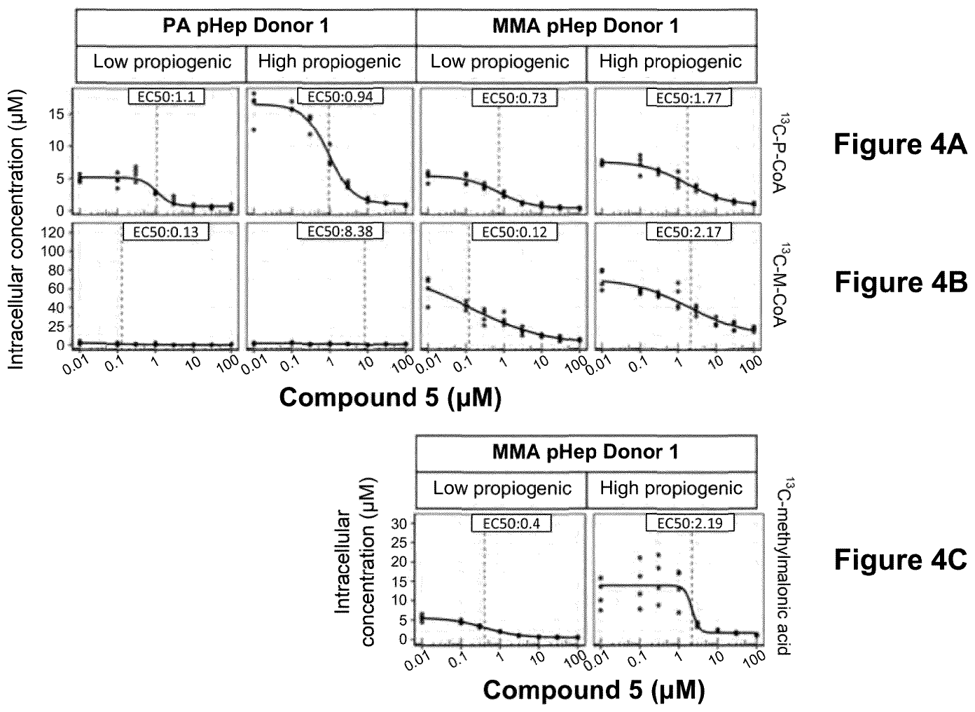

The activity of Compound 5 in primary hepatocytes (pHeps) from PA and MMA donors was also demonstrated using static cell culture experiments. Unlike the HemoShear Technology, this assay is carried out without constant perfusion, utilizing cell culture media that was customized to mimic plasma levels of propiogenic sources (amino acids and ketoacids) in PA and MMA patients during periods of relative metabolic stability (low propiogenic media) and acute metabolic crisis (high propiogenic media). PA and MMA pHeps, in static cell culture, were treated with Compound 5 (concentrations ranging from 0.1 μM to 100 μM) for 30 minutes in low propiogenic media, followed by either a continuation of low propiogenic media or a switch to the high propiogenic media for 1 hour. The media used during the 1-hour incubation contained propiogenic SIL amino acids and ketoacids which are metabolized into labeled P-CoA and M-CoA in the cells. The SIL amino acids and ketoacids were a mix of ¹³C and MeD8 labelling, but their catabolism produced a SIL P-CoA (denoted as ¹³C—P-CoA for simplicity) with the same mass, independent of the type of SIL (also true for ¹³C-M-CoA). Representative data shown in FIGS. 4A-4C illustrate that PA pHeps had a more robust increase in ¹³C—P-CoA levels in high propiogenic media, while MMA pHeps had a slight increase in ¹³C—P-CoA, no change in ¹³C-M-CoA, and an increase in methylmalonic acid (FIGS. 4A-4C).

The EC₅₀ values for Compound 5-dependent reduction in ¹³C—P-CoA and ¹³C-M-CoA were similar and independent of low vs high propiogenic media condition (Table 3). The average EC₉₀ value across all biomarkers is 11±9.6 μM. At the dose of 30 μM selected for this calculation (as described above), the percent reduction in ¹³C—P-CoA in PA and MMA pHeps exposed to low propiogenic media was −76.4±12.6% and −77.6±9.8%, respectively. When PA and MMA pHeps were exposed to high propiogenic sources to mimic a metabolic crisis, Compound 5 reduced ¹³C—P-CoA by −85.3±9.1% in PA pHeps and −75.9±7.3% in MMA pHeps. The reduction in ¹³C-M-CoA in MMA pHeps appeared to be somewhat greater under these conditions (low propiogenic: −76.5±13.2%; high propiogenic: −73±5.8%) compared to the ¹²C-M-CoA values measured in the HemoShear Technology (−55±6.6% reduction) (Table 2; Table 3). It is hypothesized that this difference is due to the lower background in ¹³C-M-CoA compared to ¹²C-M-CoA (FIG. 3B vs. FIG. 4B). The EC₉₀ values for reduction of P-CoA and M-CoA are closely matched in the different experimental designs and media formulations. These results indicate that the metabolic pathways do not deteriorate during the very short duration of the static culture experiments.

TABLE 3
PA and MMA Biomarker Levels and EC₅₀ and EC₉₀ Values of Compound
5 in Low and High Propiogenic Media in Static Cell Culture

| | Disease | | % change at | Curve | | |
|---|---|---|---|---|---|---|
| Biomarker | pHeps | Media | 30 μM | count | EC₅₀ μM | EC₉₀ μM |
| ¹³C-Propionyl-CoA | PA | low | −76.4 ± 12.6 | 5 | 2 ± 1.8 | 6.9 ± 5.4 |
| (¹³C-P-CoA) | MMA | propiogenic | −77.6 ± 9.8 | 6 | 1.3 ± 0.6 | 15.7 ± 18 |
| ¹²C-Methylmalonyl-CoA | PA | | NA | NA | NA | NA |
| (¹³C-M-CoA) | MMA | | −76.5 ± 13.2 | 5 | 2.9 ± 1.6 | 30.9 ± 16.2 |
| ¹³C-Methymalonic acid | PA | | NA | NA | NA | NA |
| | MMA | | −83.2 ± 5 | 3 | 1.2 ± 1.3 | 5.2 ± 3.3 |
| ¹³C-Propionyl-CoA | PA | high | −85.3 ± 9.1 | 6 | 1.7 ± 1 | 11.6 ± 7.4 |
| (¹³C-P-CoA) | MMA | propiogenic | −75.9 ± 7.3 | 6 | 1.6 ± 0.8 | 48.2 ± 47.5 |
| ¹²C-Methylmalonyl-CoA | PA | | NA | NA | NA | NA |
| (¹³C-M-CoA) | MMA | | −73 ± 5.8 | 4 | 2.7 ± 1.6 | 21.2 ± 2 |
| ¹³C-Methymalonic acid | PA | | NA | NA | NA | NA |
| | MMA | | −91 ± 4 | 6 | 1.1 ± 0.9 | 4.6 ± 3 |

Values are average ± standard deviation
NA—Not applicable

### Example 6

### Proposed Mechanism of Action of Compound 5 in Treating MMA and PA

Without being bound by any particular theory, it is believed the mechanism of action for Compound 5 involves the metabolism of Compound 5 in a similar manner to that of small to medium chain fatty acids. For example, Compound 5 can be biotransformed into 2,2-dimethylbutyryl-CoA, also referred to as Compound 5-CoA. This reaction utilizes CoASH. The subsequent metabolism of Compound 5-CoA by β-oxidation would be reduced because Compound 5 does not a have a proton on the alpha carbon, which prevents it from being a substrate for an acyl-CoA dehydrogenase. It is hypothesized that Compound 5 drives a redistribution of the acyl-CoA pools resulting in a reduction of intracellular levels of toxic P-CoA and M-CoA, along with a concomitant lowering of the C3/C2 acyl carnitine ratio and related organic acid metabolites (methylmalonic acid and MCA). This effect on P-CoA levels may be the result of either slowed production or enhanced clearance or a combination of these effects.

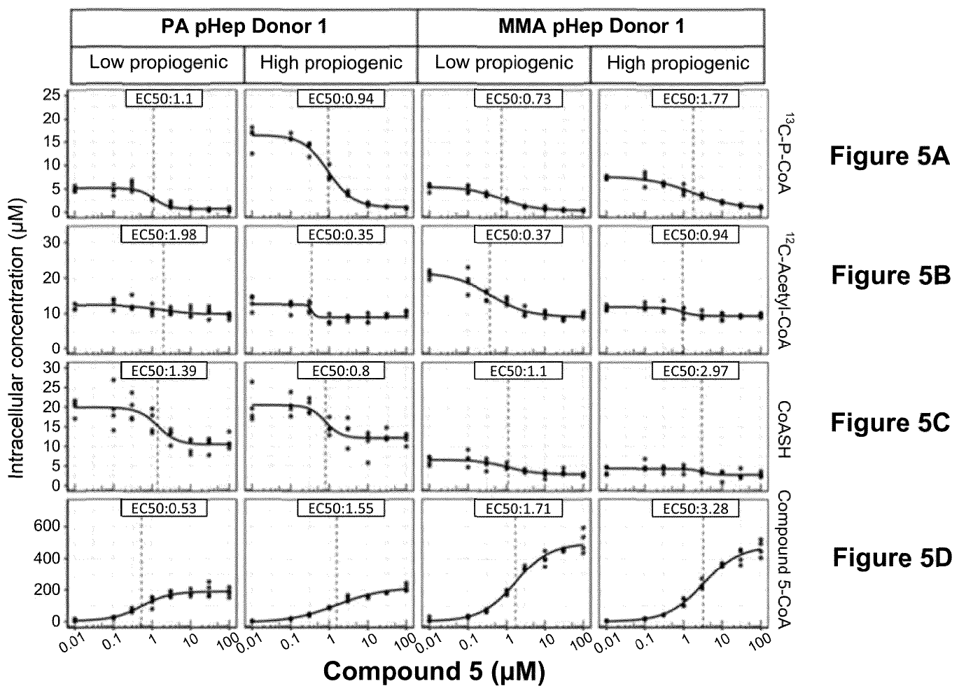

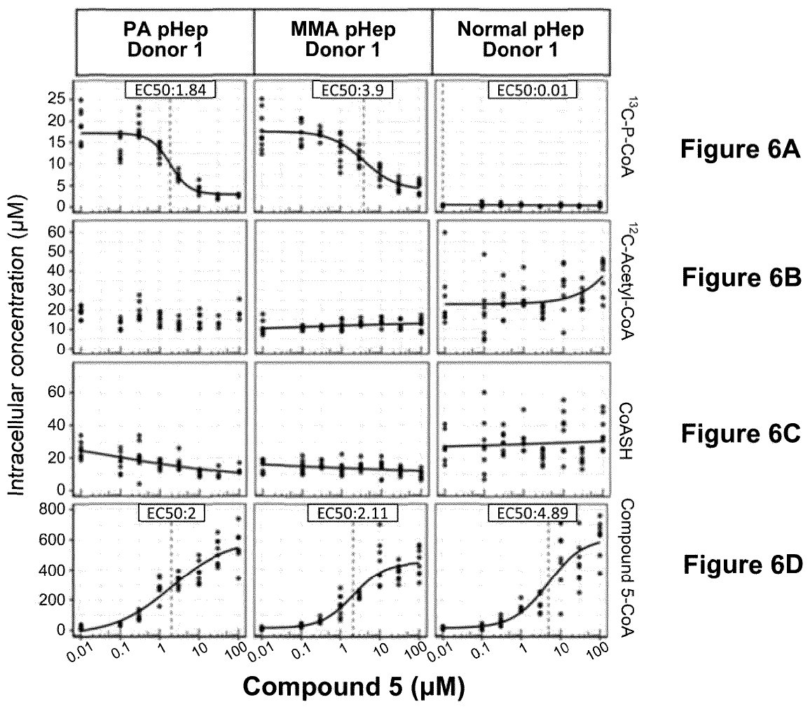

In representative data from PA and MMA pHeps (FIG. 5D; FIG. 6D), the formation of Compound 5-CoA is dose-dependent and similar, whether cells were exposed to Compound 5 over 1.5 hours or 6 days. Importantly, the EC₅₀ value for Compound 5-CoA production correlates with the EC₅₀ value for reduction of P-CoA (FIG. 5A and FIG. 5D; FIG. 6A and FIG. 6D).

In PA and MMA pHeps, where P-CoA and M-CoA levels are very high, there is a dramatic reduction in P-CoA and M-CoA pools with Compound 5 treatment (Table 2 and Table 3). Changes observed with other acyl-CoAs are not as dramatic, suggesting a target specificity that is relevant to the metabolites that accumulate in PA and MMA. The effect of Compound 5 on levels of acetyl-CoA were measured in PA and MMA pHeps in acute static experiments and in PA, MMA and normal pHeps following chronic exposures in the HemoShear Technology (FIG. 5B; FIG. 6B). In static pHeps, there is a partial dose-dependent reduction in acetyl-CoA with acute exposure to Compound 5 (FIG. 5B). Overall, there was significant variability in acetyl-CoA levels following Compound 5 treatment in the HemoShear Technology (FIG. 6B; Table 3). The data illustrate that acetyl-CoA levels were increased in PA, MMA, and normal pHeps, but there was a high SD across the percent change. Although the acetyl-CoA data from the HemoShear Technology were inconclusive, they do not show a decrease in acetyl-CoA as observed in static cell culture experiments (Table 3; Table 4). This may suggest that with Compound 5 treatment over time, acetyl-CoA is not a preferentially targeted acyl-CoA compared P-CoA and M-CoA.

TABLE 4
Compound 5 Pharmacology in the HemoShear Technology

| | | Percent | | | |
|---|---|---|---|---|---|
| | Disease | change | Curve | | |
| Analyte | pHeps | at 30 μM | count | EC₅₀ μM | EC₉₀ μM |
| ¹²C-Acetyl- | PA | 54 ± 80.6 | 3 | 2.3 ± 1.9 | 14.4 ± 13.6 |
| CoA | MMA | 13.5 ± 24.9 | 0 | NC | NC |
| | Normal | 20 ± 2.9 | 0 | NC | NC |
| CoASH | PA | −31.3 ± 24.2 | 2 | 11.6 ± 1.2 | 29.8 ± 24.3 |
| | MMA | −22.3 ± 22.3 | 2 | 14.1 ± 8.4 | 23.8 ± 20.2 |
| | Normal | −35.2 ± 3 | 0 | NC | NC |
| Compound | PA | NC | 6 | 3 ± 1.5 | 47.1 ± 39.7 |
| 5-CoA | MMA | NC | 6 | 3.5 ± 1.7 | 52.4 ± 45.6 |
| | Normal | NC | 2 | 8.3 ± 8 | 26.1 ± 3.1 |

Values are average ± standard deviation
NC - Value could not be calculated

To further evaluate the hypothesis that Compound 5 drives a redistribution of the acyl-CoA pools resulting in a reduction in P-CoA and M-CoA, the levels of CoASH were measured in the PA and MMA disease models. In 1.5 hour long static experiments utilizing PA and MMA pHeps, with either low and high propiogenic media, Compound 5 partially reduced CoASH levels with an EC₅₀ value similar to the EC₅₀ values for the reduction in P-CoA and production of Compound 5-CoA (FIG. 5C, Table 5). The effect of Compound 5 on CoASH is less pronounced in PA and MMA pHeps exposed to Compound 5 for 6 days in the HemoShear Technology (FIG. 6C; Table 3). Though in general there is a trend towards a slight decrease in CoASH with increasing dose of Compound 5, the change is statistically significant in less than half of the individual PA and MMA donors. Because many of the curves did not pass quality control, the EC₅₀/EC₉₀ values could be calculated for only 1 PA and 1 MMA donor (Table 3). Of note, the calculated EC₅₀ value for CoASH is 10-fold higher than the EC₅₀ value for the reduction in disease biomarkers (Table 2, Table 3). The Compound 5 mechanism of action is not unique to PA and MMA pHeps. Normal pHeps exposed to Compound 5 in the HemoShear Technology produce Compound 5-CoA and show a reduction in CoASH that is similar to PA and MMA pHeps (FIGS. 6A-6D, Table 3). The mechanism of action is thought to be consistent regardless of the experimental condition. Results suggest that pHeps chronically exposed to Compound 5 treatment in the HemoShear technology either recover or adapt from the larger changes in CoASH levels observed following acute Compound 5 exposures in static pHeps. Thus, in the more physiologically relevant system, changes in CoASH are slight compared to the robust reductions in P-CoA and M-CoA.

TABLE 5
Compound 5 Pharmacology in Static Culture

| | | | Percent | | | |
|---|---|---|---|---|---|---|
| | Disease | | change | Curve | | |
| Analyte | pHeps | Media | (30 μM) | count | EC₅₀ μM | EC₉₀ μM |
| ¹²C-Acetyl- | PA | low | −36.6 ± 13.5 | 4 | 0.4 ± 0.2 | 1.6 ± 1.3 |
| CoA | MMA | propiogenic | −35.5 ± 18.1 | 6 | 2.5 ± 2.6 | 28.4 ± 52 |
| CoASH | PA | | −55.2 ± 7.1 | 5 | 0.9 ± 0.3 | 9.1 ± 5.8 |
| | MMA | | −47.7 ± 13.4 | 5 | 2.3 ± 1.6 | 14.2 ± 13.5 |
| Compound 5- | PA | | NC | 5 | 1.4 ± 0.6 | 15.3 ± 11.3 |
| CoA | MMA | | NC | 6 | 2 ± 0.7 | 18 ± 15.7 |
| ¹²C-Acetyl- | PA | high | −27 ± 3.4 | 3 | 0.7 ± 0.5 | 1.9 ± 1.3 |
| CoA | MMA | propiogenic | −14.6 ± 12.6 | 2 | 1.4 ± 1.4 | 6.4 ± 7.1 |
| CoASH | PA | | −48 ± 14.3 | 4 | 2.1 ± 1.6 | 11.1 ± 13 |
| | MMA | | −28.7 ± 7.3 | 2 | 13.1 ± 14.8 | 65.6 ± 81.2 |
| Compound 5- | PA | | NC | 6 | 2.6 ± 1.6 | 42 ± 45.8 |
| CoA | MMA | | NC | 6 | 5.4 ± 2.8 | 80.1 ± 57 |

Values are average ± standard deviation
NC—Value could not be calculated

CoA sequestration has been proposed to be associated with toxicity in many disorders of intermediary metabolism, including PA and MMA. It is hypothesized that sequestration of CoASH into accumulating P-CoA and M-CoA leads to a reciprocal decrease in acetyl-CoA and/or CoASH; however, the idea has little to no supporting evidence due to the inability to measure and study tissue acyl-CoA and CoASH levels in humans. While some effect on acetyl-CoA and CoASH was observed, particularly in static culture conditions, these effects were not as pronounced as those observed on other metabolites.

### CONCLUSIONS

The studies described herein demonstrated that Compound 5 reduces the toxic metabolites P-CoA, M-CoA, C3, MCA, and methylmalonic acid (MMA only) in pHep-based disease models of PA and MMA. Overall, the EC₉₀ values for reduction of biomarker concentrations were consistent across all the biomarkers, suggesting that Compound 5 has an effect on correcting relevant metabolic abnormalities in PA and MMA that is consistent with the biochemical pathways thought to underlie the disease pathology and thus supports its therapeutic potential in both disorders. Since the compounds of the disclosure are able to form CoA esters, these compounds can also treat diseases characterized by the buildup of toxic levels of the metabolites described herein by redistribution of the acyl-CoA pools.

### Example 7

Phase 2 Study of Compound 5A in Subjects with Propionic or Methylmalonic Acidemia

### ABBREVIATION LIST

| | |
|---|---|
| ADHD | attention deficit, hyperactivity disorder |
| AE | adverse event |
| ALK | alkaline phosphatase |
| ALT | alanine aminotransferase |
| AST | aspartate aminotransferase |
| AUC | area under the concentration time curve |
| BSID-IV | Bayley Scales of Infant and Toddler Development, |
| | Fourth Edition |
| BID | twice daily |
| C2 | acetyl-carnitine |
| C3 | propionyl-carnitine |
| C24 | concentration at 24-hr post-dose |
| CA | citric acid |
| CBC | complete blood count |
| CblA | cobalamin A |
| CblB | cobalamin B |
| CI | confidence interval |
| CL/F | clearance |
| CFR | Code of Federal Regulations |
| Cₘₐₓ | maximum concentration |
| Cₘᵢₙ | minimum concentration |
| CNS | central nervous system |
| CoASH | coenzyme A |
| Cₚᵣₑ | pre-dose concentration |
| CRF | Case Report Form |
| CTCAE | Common Terminology Criteria for Adverse Events |
| DDI | Drug-Drug Interaction |

### ABBREVIATION LIST

| | |
|---|---|
| DMC | Data Monitoring Committee |
| DNA | Deoxyribonucleic Acid |
| DRE | Disease-Related Events |
| EC90 | Effective Concentration 90% |
| ECG | electrocardiogram |
| ECHO | echocardiogram |
| eCRF | electronic case report form |
| eGFR | estimated glomerular filtration |
| FAS | Full Analysis Set |
| FDA | Food and Drug Administration |
| FGF21 | fibroblast growth factor 21 |
| FSH | follicle stimulating hormone |
| GCP | Good Clinical Practice |
| GGT | gamma-glutamyl transpeptidase |
| GI | gastrointestinal |
| HIV | human immunodeficiency virus |
| HRQOL | Health-related Quality of Life |
| HRT | hormonal replacement therapy |
| IB | Investigator's Brochure |
| ICF | Informed Consent Form |
| ICH | International Council on Harmonization |
| IEC | Independent Ethics Committee |
| INR | international normalized ratio |
| IRB | Institutional Review Board |
| IVRS | Interactive Voice Response System |
| IWRS | Interactive Web Response System |
| LC-MS/MS | liquid chromatography with tandem mass spectrometry |
| LVEF | left ventricular ejection fraction |
| MAR | missing at random |
| M-CoA | methylmalonyl-coenzyme A |
| MCA | 2-methylcitric acid |
| MedDRA | Medical Dictionary for Regulatory Activities |

### ABBREVIATION LIST

| | |
|---|---|
| MMA | methylmalonic acidemia |
| MMRM | mixed effect model repeated measures |
| MRI | magnetic resonance imaging |
| MUT | methylmalonyl-CoA mutase |
| NAGS | N-acetylglutamate synthase |
| NBIA | Neurodegeneration with Brain Iron Accumulation |
| NCA | non-compartmental analysis |
| NOAEL | No-observed-adverse-effect level |
| OAA | oxaloacetic acid |
| OL-LTE | open-label, long-term extension |
| PA | propionic acidemia |
| PCC | propionyl-CoA carboxylase |
| P-CoA | propionyl-coenzyme A |
| PD | pharmacodynamics |
| pg | per gastrostomy tube |
| PK | pharmacokinetics |
| po | per orum (oral) |
| QD | once daily |
| QOL | quality of life |
| QTcF | QTc interval calculated using Fridericia's method |
| RI | run-in |
| SRC | Safety Review Committee |
| SAE | serious adverse event |
| SAP | Statistical Analysis Plan |
| SD | standard deviation |
| SoA | Schedule of Assessments |
| SoC | standard of care |
| SAP | statistical analysis plan |
| SFA | stature for age |
| TCA | tricarboxylic acid |
| TEAE | treatment-emergent adverse event |
| Tₘₐₓ | time of maximum concentration |

### ABBREVIATION LIST

| | |
|---|---|
| t½ | half-life |
| ULN | upper limit of normal |
| US | United States |
| Vz/F | volume of distribution |
| WASI-II | Wechsler Abbreviated Scale of Intelligence, Second Edition |
| WFA | weight-for-age |
| WPPSI-IV | Wechsler Preschool and Primary Scale of Intelligence, |
| | Fourth Edition |
| WHO | World Health Organization |
| WOCBP | Woman of Childbearing Potential |
| λz | terminal elimination rate |

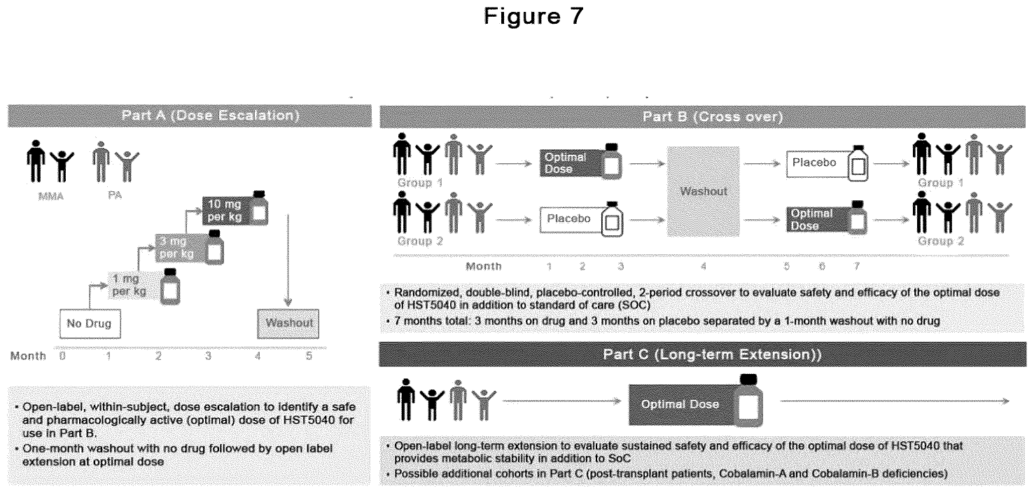

Summary of Three-Part Phase 2 Protocol Evaluating Compound 5A (2,2-Dimethylbutyric Acid Sodium Salt) in Subjects with Propionic or Methylmalonic Acidemia (FIG. 7)

Title of Study: A Phase 2, Open-label, Dose Escalation Study of Compound 5 in Subjects with Propionic or Methylmalonic Acidemia Followed by 6-Month, Randomized, Double-blind, Placebo-controlled, 2-period Crossover Study and Open-label, Long-term Extension Study.

Condition/Disease: Propionic Acidemia (PA) or Methylmalonic Acidemia (MMA).

Number of Subjects: A minimum of 12 subjects (≥2 years old) participated in the study. Each part of the study included approximately 6 PA (targeting approximately 3 younger subjects between the ages of 2-11 and 3 older subjects ≥12) and 6 MMA (targeting approximately 3 younger subjects between the ages of 2-11 and 3 older subjects ≥12) subjects. Subjects in Part B participated in Part A, and the Part A extension of the study. Subjects in Part C will have participated in Part B of the study. Additional subjects may be enrolled in Part B or C of the study if there are dropouts in Part A, the Part A extension, or Part B, or if the Sponsor in consultation with the data monitoring committee (DMC) determines that additional subjects are needed. In addition, additional cohorts of up to 6 evaluable subjects each with MMA associated cobalamin A (cblA) or cobalamin B (cblB) or liver and/or kidney transplant may be considered in Part C.

### Duration of Subject Participation:

Part A: minimum of 6 months
Part A Extension: up to several months
Part B: 7 months
Part C: Until marketing authorization or until Sponsor discontinues the study

### Duration of Study (Including Estimates of Subject Enrollment):

Part A (within-subject dose escalation): The minimum planned duration is 11 months including approximately 5 months recruitment time; screening (approximately 1 month); run-in period (1 month); dose escalation (3 months); washout (1 month).
Part A Extension: Subjects completing Part A dose escalation will continue to receive Compound 5 in Part A at their highest tolerated dose until the optimal dose of Compound 5 has been determined.
Part B (6-month, randomized, double-blind, placebo-controlled, 2-period crossover): The duration of each subject's participation is 7 months including 2 periods of 12-weeks duration each and 4-week washout in between periods.
Part C (open-label, long-term extension; OL-LTE): Up to 5 years duration. Enrollment in Part C will begin after internal review of available safety, PK, and efficacy data at the completion of Part B and participation will continue until market authorization or discontinuation of study.

### Study Design Description: This is an Interventional Study to Assess the Safety, PK, and Efficacy of Compound 5. The Study Consists of 3 Parts:

Part A: open-label, within-subject, dose escalation study in PA and MMA subjects ≥2 years old (N=approximately 12, 6 each targeting approximately 3subjects ages 2-11 and 3 subjects ≥12) to identify a safe and pharmacologically active (optimal) dose of Compound 5 for use in Part B.
Part A Extension: open-label extension therapy for PA and MMA subjects ≥2 years old who have completed dose escalation in Part A until the optimal dose of Compound 5 is identified for use in Part B.
Part B: 6-month, randomized, double-blind, placebo-controlled, 2-period crossover with 4-week washout between treatment periods in PA and MMA subjects ≥2 years old (N=approximately 12, 6 each) to evaluate safety and efficacy of the optimal dose of Compound 5 in addition to standard of care (SoC).
Part C: open-label long-term extension study in PA and MMA subjects ≥2 years old (N=approximately 12, 6 each) to evaluate the long-term safety and efficacy of the optimal dose of Compound 5 that provides metabolic stability.

Study assessments included standard safety assessments (physical examination, vital signs, clinical laboratory values, AEs, ECG, Holter monitor, and ECHO) as well as monitoring for signs and symptoms of worsening disease (e.g., increased frequency of metabolic decompensations, lethargy, food intolerance, encephalopathy, or progressive organ dysfunction) or free CoASH deficiency (clinical manifestations similar to CoASH biosynthetic defects that cause Neurodegeneration with Brain Iron Accumulation (NBIA), i.e., iron accumulation in the basal ganglia detectable by T2-weighted magnetic resonance imaging [MRI] and an extrapyramidal movement syndrome characterized by dystonia, spasticity, and Parkinsonism). Efficacy assessments included PA and MMA disease-related biomarker with added assessment of ureagenesis (Part B only), and clinical outcomes assessments including number of episodes and days requiring hospitalization, an ER visit, or use of a home emergency treatment protocol for metabolic decompensations, hematologic abnormalities, cardiac disease, oral intake, neurocognitive assessments, and quality of life, including parent quality of life and family functioning, and subject- and Clinician-reported global impressions of severity and change.

### Part A—Safety, PK, and Proof of Concept (Open-Label, Dose Escalation Study) of Compound 5 in PA and MMA Subjects

Primary Objectives: To determine the optimal dose of Compound 5 in PA and MMA subjects ≥2 years old:
Safety: Evaluate the safety of 3 dose levels of Compound 5 administered orally or per gastrostomy tube once daily (po/pg QD) in a minimum of 12 subjects with PA or MMA (6/6) for a minimum of 4 weeks at each dose level per subject:
   Treatment-emergent adverse events (TEAEs) associated with each dose level (incidence, severity, seriousness, and relatedness); within-subject change and/or shift from baseline to end of each dose level interval in physical examination, vital signs, and clinical laboratories; triplicate electrocardiogram (ECG) rate, intervals, and arrhythmias
   Safety follow-up of individual subjects initially enrolled and treated in Part A before proceeding with next dose level in Part A for any untoward events, in particular signs and symptoms of worsening disease or free coenzyme A (CoASH) deficiency
Pharmacokinetics (PK): Evaluate the systemic exposure to support the dose and dosing regimen of Compound 5:
   PK: maximum concentration (Cₘₐₓ), time of maximum concentration (Tₘₐₓ), area under the concentration time curve (AUC), minimum concentration (Cₘᵢₙ), terminal elimination rate (λ_{z}), half-life (t_{1/2}), volume of distribution (Vz/F) and clearance (CL/F)
   Compound 5 concentrations (including Cₚᵣₑ during the Part A Extension)
Efficacy: Evaluate the Pharmacodynamic (PD) response to Compound 5 in PA and MMA subjects through improvements in the 2-methylcitric acid (MCA) level:
   Within-subject percent change from baseline to end of each dose level interval in plasma MCA (fasting for minimum of 3 hours)

### Secondary Objectives:

Evaluate the PD response to Compound 5 in PA and MMA subjects through improvements in other disease-related biomarkers and tricarboxylic acid cycle function (TCA):
   Within-subject change from baseline to end of each dose level interval in TCA cycle function using plasma MCA: citric acid (MCA: CA) ratio.
   Within-subject change from baseline to end of each dose level interval in disease-related biomarker panel: plasma propionyl-carnitine (C3), C3 to acetyl-carnitine ratio (C3: C2), 3-OH propionate, methylmalonic acid (in MMA subjects), NH3 (minimum 3 hours fasting), anion gap, venous pH, amino acids, carnitine (total, free, and esterified), ketones (β-OH butyrate), lactic acid; urine ketones, organic acids, and acylglycines.
Evaluate the total number of metabolic decompensation events requiring an emergency room (ER) visit or hospitalization:
   Frequency of episodes requiring hospitalization or an ER visit for metabolic decompensations, overall and per dose level interval.
Evaluate the total number of hospital days required for treatment/resolution of metabolic decompensation:
   Frequency of days hospitalized or visited the ER for metabolic decompensations, overall and per dose level interval.
Evaluate the total number of episodes and days requiring use of a home emergency treatment protocol for metabolic decompensations:
   Frequency of episodes and frequency of days requiring use of a home emergency treatment protocol for metabolic decompensations, overall and per dose level interval.
Evaluate changes to hematologic abnormalities:
   Within-subject change from baseline to end of each dose level interval in complete blood cell (CBC) count with differential: absolute neutrophil count (#/10⁻⁹L), platelet count (#/10⁻⁹L), hemoglobin level (g/dL).
Evaluate changes in the route of dietary intake:
   Within-subject change from baseline to end of each dose level interval in oral intake (percent of daily calories oral vs. gastrostomy tube).
   Within-subject change from baseline to end of each dose level interval in assessment of diet (total, natural, and medical protein intake in g/kg/day; total caloric intake in Kcal/kg/day).
Evaluate the effects of Compound 5 on the QTc interval:
   Within-subject change from baseline to end of each dose level interval in QTc interval calculated using Fridericia's method (QTcF) (msec) interval by Holter monitor and triplicate ECG: percent of subjects with normal QTc intervals.
   QTcF interval-Compound 5 plasma concentration response relationship for time-matched collections.
Evaluate improvement in Health-related Quality of Life (HRQOL) and global assessment:
   Change from baseline to week 16 in MetabQoL 1.0 (Total, Physical, Mental, and Social Scores).
   Subject/Caregiver- and Clinician-Reported Global Assessments of Severity and Change Questionnaire scores.
   Change from baseline to week 16 in PedsQL™ Family Impact Module (Total, Parent HRQOL, and Family Functioning Scores).
Evaluate improvement in cardiac disease:
   Change from baseline to end of each dose level interval in left ventricular ejection fraction (LVEF) by echocardiogram (ECHO).
Evaluate whether PD biomarkers return to baseline levels during the washout period:
   Washout Primary Endpoints: Within-subject percent change from baseline and week 16 to weeks 18 and 20 in plasma MCA (fasting for minimum of 3 hours)
   Washout Secondary Endpoints: Within-subject percent change from baseline and week 16 to weeks 18 and 20 in disease-related biomarker panel: plasma MCA: CA ratio, C3, C3: C2 ratio, 3-OH propionate, methylmalonic acid (in MMA subjects), anion gap, venous pH, amino acids, carnitine (total, free, and esterified), ketones ((3-OH butyrate), lactic acid, urine ketones, organic acids, and acylglycines.

### Exploratory Endpoints:

Evaluate urinary Compound 5 excretion and plasma and urine metabolites of Compound 5:
   Urinary levels of Compound 5 and plasma and urinary levels of Compound 5 metabolites (including, but may not be limited to acylcarnitine, acylglycine, and acylglucuronide metabolites).
Evaluate the PD response to Compound 5 in PA and MMA subjects through improvements in other disease-related biomarkers:
   Within-subject change from baseline to end of each dose level interval in plasma fibroblast growth factor 21 (FGF21).

### Protocol Part A: Within-Subject Dose Escalation and Open-Label Extension:

Part A is the dose escalation phase to assess the initial safety and to confirm the pharmacologic activity of Compound 5 in subjects with PA and MMA. Screening for eligibility took place within 28 days of enrollment. All subjects were initially evaluated during a 4-week run-in period followed by within-subject dose escalation of 3 doses (1, 3, and 10 mg/kg QD of sodium 2,2-dimethylbutanoate); dose escalation interval of 4 weeks) to check for improvement in disease-related biomarkers.

All available safety, PK, and efficacy data was reviewed internally during within-subject dose escalation by an internal Safety Review Committee (SRC). Additionally, an independent Data Monitoring Committee (DMC) reviewed all available safety, PK, and efficacy data prior to the start of Part B of the study. If 2 subjects experience the same Grade 3 or higher AE (based on the Common Terminology Criteria for Adverse Events version 5.0 [CTCAE v5.0]) or any single subject experiences a Grade 4 or higher AE, either of which, in the opinion of the Investigator, are considered to be related to Compound 5, enrollment and dosing of additional subjects stopped and an ad hoc DMC meeting convened to provide recommendations for the Sponsor's consideration regarding continuation, modification, or discontinuation of dosing. Subjects who do not complete dose escalation were replaced.

At the end of Part A, subjects continued to be monitored in the study for an additional 4 weeks without Compound 5 drug treatment, according to the schedule of assessments. The purpose of this washout period was to determine the time for disease-related biomarkers to return to baseline levels, and thus, the duration of the carry-over effect of pharmacologic activity on disease-related biomarkers. If this evaluation showed that the duration of the washout period needed to be altered, the crossover washout in Part B was adjusted (see below). This information is informative for the design of future studies.

After individual subjects completed the washout period, they received Compound 5 at their highest tolerated dose in an open-label extension of Part A. Monthly PK/PD and safety assessments were conducted according to the schedule of assessments. This extension of treatment continued until all subjects completed the dose escalation, the data was analyzed, and the optimal dose of Compound 5 determined for Parts B and C.

Based on the ongoing review of PK/PD data, a higher daily dose or a twice-daily dosing (BID) regimen not to exceed 30 mg/kg QD or 15 mg/kg BID of sodium 2,2-dimethylbutanoate), may be considered during the open-label extension of Part A and in Parts B/C if supported by preclinical safety and clinical PK/PD/safety data.

Part B-Safety and Efficacy of Compound 5 in PA and MMA Subjects (6-Month, Randomized, Double-blind, Placebo-controlled, 2-period Crossover)

Primary Endpoint: Evaluate the safety and efficacy at the optimal dose determined in Part A (within-subject dose escalation).
Safety: Evaluate the safety of Compound 5 in PA and MMA subjects:
   TEAEs (incidence, severity, seriousness, and relatedness); within-subject change and/or shift from baseline to end of each treatment period in physical examination, vital signs, and clinical laboratories; ECG rate, intervals, and arrhythmias
   Signs and symptoms of worsening disease or free CoASH deficiency.
Efficacy: Evaluate the PD response to Compound 5 in PA and MMA subjects through improvements in MCA levels:
   Within-subject percent change from baseline (the last measurement prior to the first dose of study drug in each treatment period) to end of each treatment period in plasma MCA (fasting for minimum of 3 hours)

### Secondary Endpoint:

Evaluate the PD response to Compound 5 in PA and MMA subjects through improvements in other disease-related biomarkers, TCA function, and ureagenesis:
   Within-subject change from baseline to end of each treatment period in MCA, MCA: CA ratio, C3, C3: C2 ratio, 3-OH propionate, methylmalonic acid (in MMA subjects), anion gap, venous pH, amino acids, carnitine (total, free, and esterified), ketones (β-OH butyrate), amino acids, and lactic acid, urine ketones, NH₃ (minimum 3 hours fasting), organic acids, and acylglycines.
   Ureagenesis using 13C-acetate at the end of each treatment period.
Evaluate the total number of metabolic decompensation events requiring an ER visit or hospitalization:
   Frequency of episodes requiring hospitalization, or an ER visit for metabolic decompensations, per treatment period.
Evaluate the total number of hospital days and ER visits required for treatment/resolution of metabolic decompensation:
   Frequency of days hospitalized or visited the ER for metabolic decompensations, per treatment period.
Evaluate the total number of episodes and days requiring use of a home emergency treatment protocol for metabolic decompensations:
   Frequency of episodes and frequency of days requiring use of a home emergency treatment protocol for metabolic decompensations, per treatment period.
Evaluate improvements in hematologic abnormalities:
   Within-subject change from baseline to end of each treatment period in CBC count with differential: absolute neutrophil count (#/10⁻⁹L), platelet count (#/10⁹ L), hemoglobin level (g/dL).
Evaluate changes in the route of dietary intake:
   Within-subject change from baseline to end of each treatment period in oral intake (percent of daily calories oral vs. gastrostomy tube).
   Within-subject change from baseline to end of each treatment period in assessment of diet (total, natural, and medical protein intake in g/kg/day; total caloric intake in Kcal/kg/day).
Evaluate improvements in cardiac disease:
   Within-subject change from baseline to end of each treatment period in QTcF interval by ECG (msec): percent of subjects with normal QTc intervals; percent of subjects with increases in QTcF to >500 msec; and percent of subjects with increases of >60 msec over baseline.
   Change from baseline in LVEF by ECHO.
Evaluate improvements in HRQOL and global assessment:
   MetabQoL 1.0 (Total, Physical, Mental, and Social Scores) at the end of each treatment period
   Subject/Caregiver- and Clinician-Reported Global Assessments of Severity and Change scores at the end of each treatment period.
   PedsQL™ Family Impact Module (Total, Parent HRQOL, and Family Functioning Scores) at the end of each treatment period.
Evaluate improvement in neurocognitive and developmental function:
   Change from Baseline (Part A) at the end of each treatment period in scores on Wechsler Abbreviated Scale of Intelligence, Second Edition (WASI-II).
   Change from Baseline (Part A) at the end of treatment period in scores on Wechsler Preschool and Primary Scale of Intelligence, Fourth Edition, (WPPSI—IV).
   Change from Baseline (Part A) at the end of each treatment period in scores on Bayley Scales of Infant and Toddler Development, Third Edition (BSID-IV).
Characterize the PK of Compound 5:
   PK: Compound 5 plasma concentrations (may be combined with Part A for population PK and covariate analyses).

### Exploratory Endpoints:

Evaluate the PD response to Compound 5 in PA and MMA subjects through improvements in other disease-related biomarkers:
   Within-subject change from baseline to end of each dose level interval in plasma FGF21.

### Protocol Part B: 6-Month, Randomized, Double-Blind, Placebo-Controlled, 2-Period Crossover Study:

Part B is the 6-month, randomized, double-blind (Subject/Investigator/Sponsor), placebo-controlled, 2-period crossover study consisting of 2 intervention periods of 12 weeks each separated by a washout period of at least 4 weeks to evaluate the safety and efficacy of the optimal dose of Compound 5 in PA and MMA subjects ≥2 years old (N=minimum 12) in addition to SoC determined in Part A (within-subject dose escalation).

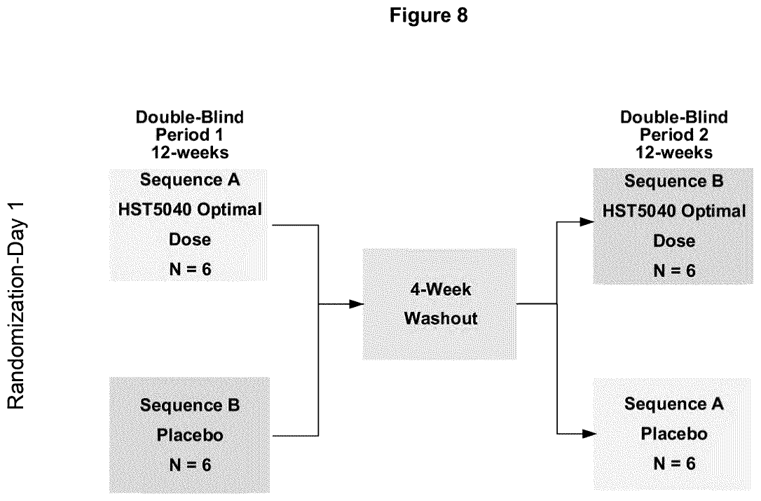

Subjects must have completed Part A and the Part A open-label extension to participate in Part B unless the DMC and Sponsor determine that the replacement of discontinued subjects from Parts A or B is warranted to further evaluate the safety, PK, or PD of Compound 5. Once the optimal dose of Compound 5 has been determined, all subjects will initiate Part B of the study. Subjects will be randomly_assigned to treatment sequence, stratified by disease type to ensure a balance of sequences within each disease type. If a subject drops out early and is not part of the crossover evaluable set, the subject will be replaced. Prior to initiating Part B, any replacement subjects will be required to undergo a 4-week run-in period as described for Part A. The same measures will be collected multiple times for each subject during each period allowing the evaluation of safety and efficacy measures. The Sponsor could add new subjects in Part B if further evaluation of safety, PK, or PD of Compound 5 is warranted or if advised by the DMC. A schematic of the study design for Part B showing the 2-way period crossover is shown in FIG. 8.

If 2 subjects experience the same Grade 3 or higher adverse event (AE; based on the CTCAE v5.0) or any single subject experiences a Grade 4 or higher AE, either of which, in the opinion of the Investigator, are considered to be related to Compound 5, enrollment and dosing of additional subjects will be stopped and an ad hoc DMC meeting will be convened to provide recommendations for the Sponsor's consideration regarding continuation, modification, or discontinuation of dosing.

Available safety, PK, and efficacy data will be reviewed internally at the completion of Part B and prior to subjects initiating Part C. In addition to standard safety assessments, subjects will be monitored for signs and symptoms of worsening disease or free CoASH deficiency. A DMC meeting will be convened at the end of Part B to review all available safety, PK and efficacy data, but the initiation of Part C will not be contingent upon the meeting. Thereafter, DMC meetings will convene every 6 months, at minimum.

### Part C—Safety and Efficacy of Compound 5 in PA and MMA Subjects (Open-Label, Long-Term Extension)

### Primary Endpoints:

Evaluate the long-term safety and sustained efficacy following treatment in Part B (6 month, randomized, double-blind, placebo-controlled, 2-period crossover):
   TEAEs (incidence, severity, seriousness, and relatedness); within-subject change and/or shift from baseline to end of each year and end of study in physical examination, vital signs, and clinical laboratories; ECG rate, intervals, and arrhythmias.
   Signs and symptoms of worsening disease or free CoASH deficiency.
Evaluate the total number of metabolic decompensation events requiring an ER visit or hospitalization:
   Frequency of episodes requiring hospitalization, or an ER visit for metabolic decompensations overall and per year.
Evaluate the total number of hospital days and ER visits required for treatment/resolution of metabolic decompensation:
   Frequency of days hospitalized or visited the ER for metabolic decompensations, overall and per year.

### Secondary Endpoints:

Evaluate the total number of episodes and days requiring use of a home emergency treatment protocol for metabolic decompensations:
   Frequency of episodes and frequency of days requiring use of a home emergency treatment protocol for metabolic decompensations, overall and per year.
Evaluate the PD response to Compound 5 in PA and MMA subjects through improvements in disease-related biomarkers and TCA function:
   Within-subject change from baseline to end of each year and end of study in plasma MCA, MCA: CA ratio, C3, C3: C2 ratio, 3-OH propionate, methylmalonic acid (in MMA subjects), anion gap, venous pH, amino acids, carnitine (total, free, and esterified), ketones (β-OH butyrate), amino acids, and lactic acid, urine ketones, NH₃ (minimum 3 hours fasting), organic acids, and acylglycines.
Evaluate improvements in oral intake and liberalization of diet:
   Within-subject change from baseline to end of each year and end of study in oral intake (percent of daily calories oral vs. gastrostomy tube).
   Within-subject change from baseline to end of each year and end of study in assessment of diet (total, natural, and medical protein intake in g/kg/day; total caloric intake in Kcal/kg/day).
Evaluate improvements in hematologic abnormalities:
   Within-subject change from baseline to end of each year and end of study in CBC count with differential: absolute neutrophil count (#/10⁻⁹L), platelet count (#/10⁹L), hemoglobin level (g/dL).
Evaluate improvements in cardiac disease:
   Within-subject change from baseline to end of each year and end of study in QTcF interval by ECG (msec): percent of subjects with normal QTc intervals; percent of subjects with increases in QTcF to >500 msec; and percent of subjects with increases of >60 msec over baseline.
   Change from baseline at the end of each year and end of study in L VEF by ECHO.
Evaluate improvements in HRQOL and global assessment:
   MetabQoL 1.0 (Total, Physical, Mental, and Social Scores) at the end of each year and end of study.
   Subject/Caregiver- and Clinician-Reported Global Assessments of Severity and Change scores at end of each year and end of study.
   PedsQL™ Family Impact Module (Total, Parent HRQOL, and Family Functioning Scores) at the end of each year and end of study.
Evaluate improvement in neurocognitive and developmental function:
   Within-subject change from baseline (Part A) to end of each year and end of study in scores on WASI-II.
   Within-subject change from baseline (Part A) to end of each year and end of study in scores on WPPSI—IV.
   Within-subject change from baseline (Part A) to end of each year and end of study in scores on BSID-IV.
Evaluate improvements in growth in pediatric subjects (<18 years):
   Within-subject change from baseline in z-scores and percentiles for weight-for-age (WFA), and stature-for-age (SFA) (based on Centers for Disease Control and Prevention [CDC] child growth standards) in subjects <18 years of age on the date of informed consent.
Characterize the PK of Compound 5:
   PK: Compound 5A (sodium 2,2-dimethylbutanoate) plasma concentrations (may be combined with Parts A/B for population PK and covariate analyses).

### Exploratory Endpoints:

Evaluate the PD response to Compound 5 in PA and MMA subjects through improvements in other disease-related biomarkers:
   Within-subject change from baseline to end of each year and end of study in other plasma disease-related biomarkers.

### Protocol Part C: Open-Label, Long-Term Extension:

Part C is the OL-LTE study to evaluate the long-term safety and sustained efficacy of the optimal dose of Compound 5 in PA and MMA subjects ≥2 years old (N=approximately 12) in addition to SoC. Subjects must have completed Parts A and B to participate in Part C.

The Sponsor in consultation with the DMC may determine that the replacement of discontinued subjects from Parts A or B is warranted to further evaluate the safety, PK, or PD of Compound 5. Part C may include the enrollment of new cohort(s) of up to 6 subjects ≥2 years old with MMA associated with cblA or cblB deficiency and MMA or PA post-liver and/or kidney post-transplant. In addition to standard safety assessments, subjects will be monitored for signs and symptoms of worsening disease or free CoASH deficiency.

### Inclusion Criteria:

Subjects must meet the following criteria to be included in the study:
1. Confirmed diagnosis of symptomatic PA or MMA as defined by: (i) biochemical testing (marked elevations in C3, MCA, and/or methylmalonic acid with a normal homocysteine level), or (ii) enzymatic testing (deficient activity of propionyl-coenzyme A [P-CoA] carboxylase for PA and methylmalonyl-coenzyme A [M-CoA] mutase for MMA); (iii) genetic testing (biallelic mutations in propionyl-coenzyme A carboxylase subunits A or B (PCCA or PCCB) for PA and in L-methylmalonyl-coenzyme A mutase (MMUT) for MMA); and (iv) a history of metabolic decompensations. Depending on the results from Part A, subjects with MMA associated with defects in adenosylcobalamin synthesis caused by biallelic mutations in the MMAA (cblA) or MMAB (cblB) genes may be considered for enrollment in Part C.
2. Ages ≥2 years old.
3. History of inadequate metabolic control while receiving standard of care (SoC), as evidenced by plasma NH₃>50 μM on 2 occasions at least 1 week apart, plasma methylmalonic acid >150 μM (MMA only), or a metabolic decompensation requiring hospitalization and/or an ER visit (Part A only).
4. Plasma MCA concentration >3× upper limit of normal of the reference range at screening.
5. Subject should be on stable supplementation dose of carnitine for at least 1 week prior to the entry in the study and have a free carnitine level ≥10 nmol/mL.
6. Subject or subject's parent or legal guardian (if applicable) consents to participate in the study and provides informed consent prior to any study procedures being performed. If the subject is of minor age; he/she is willing to provide assent where required per local regulations, and if deemed able to do so.

### Exclusion Criteria:

Subjects meeting any of the following criteria are to be excluded:
1. Serologic evidence of human immunodeficiency virus (HIV) infection.
2. Serologic evidence of Hepatitis B or C infection.
3. Plasma vitamin B5 below lower limit of normal.
4. Moderate-to-severely impaired cardiac function with LVEF <45% by ECHO.
5. Clinically significant arrhythmia by Holter monitor.
6. Marked prolongation of QT/correct QT (QTc) interval (>450 msec by Fridericia method; QTcF); a history of additional risk factors for Torsades de pointes [e.g., heart failure, hypokalemia, family history of Long QT Syndrome]; and the use of concomitant medications that prolong the QT/QTc interval).
7. Moderate to severe chronic kidney disease with estimated glomerular filtration rate

(eGFR)<60mL/min/1.73m².
8. In the opinion of the Investigator, if the subject has any prior or ongoing medical condition (e.g., cardiovascular, neurological, metabolic diseases) or clinically significant screening laboratory value that may present a safety risk, interfere with study compliance, interfere with consistent study follow-up, or confound the interpretation of the study results.
9. Exposure to any investigational therapy, apart for a COVID-19 vaccine, within the past 6 months prior to study entry.
10. Exposure to gene therapy for PA or MMA at any time prior to study entry.
11. History of organ transplantation (Parts A and B only).
12. History of severe allergic or anaphylactic reactions to any of the components of Compound 5.
13. Pregnant or lactating women.
14. Unwilling or unable to comply with the requirements of this protocol (in the opinion of the Investigator) including, but not limited to, the presence of any condition (physical, mental, or social) likely to affect the subject's ability to return for study visits or complete required assessments.

### Concomitant Treatment:

Permitted Therapies: Continued use of SoC over-the-counter vitamins, dietary supplements or medical foods, bicitra, carnitine, metronidazole, nitrogen scavengers, and other medications, are permitted, at previously established and stable doses and regimens

### Dosage and Mode of Administration:

Compound 5 (2,2-dimethylbutanoic acid) is intended for oral (po) and gastric or nasogastric (pg) administration only and will be supplied as an oral solution containing 8.4 or 59 mg/mL Compound 5 (equivalent to 10 or 70 mg/mL Compound 5A (sodium 2,2-dimethylbutanoate), respectively).

Doses of 1, 3, and 10 mg/kg Compound 5A will be administered during the study; 1.0 mg of Compound 5A equals 0.84 mg of Compound 5. Placebo oral solution will be administered in the same manner as Compound 5A in Part B only. Compound 5 (and the placebo) will initially be administered po/pg QD, although more frequent dosing may be contemplated based on observed safety, PK, and PD

### Safety Evaluation:

TEAEs, physical examination, vital signs, clinical laboratory values, ECG, Holter monitor (Part A only), and ECHO.

Safety follow-up of individual subjects initially enrolled and treated in Part A before proceeding with next dose level in Part A for any untoward events, in particular signs and symptoms of worsening disease or free coenzyme A (CoASH) deficiency.

### Efficacy Evaluation:

PA and MMA disease-related biomarkers

TCA function

Ureagenesis (Part B only)

Total number of episodes and days requiring a hospitalization, or an ER visit for metabolic decompensations. Metabolic decompensations are defined as the presence of hyperammonemia (>50 μmol/L) and/or metabolic acidosis that is with an increased anion gap (>15 mEq/L) associated with gastrointestinal (e.g., anorexia, nausea, vomiting) and/or central nervous system (CNS) symptoms (e.g., lethargy, somnolence).

Total number of episodes and days requiring the use of a home emergency treatment protocol for metabolic decompensations. Metabolic decompensations are defined as the presence of moderate to severe ketosis by urine dipstick that are associated with gastrointestinal and/or CNS symptoms.

CBC with differential

Oral vs. gastrostomy tube intake and 3-day food diary

Cardiac LVEF and QTc interval

MetabQoL 1.0 (Total, Physical, Mental, and Social Scores)

PedsQL™ Family Impact Module (Total, Parent HRQOL, and Family Functioning Scores)

Subject/Caregiver- and Clinician-Reported Global Assessments of Severity and Change Questionnaire

Wechsler Abbreviated Scale of Intelligence, Second Edition (WASI-II)

Bayley Scales of Infant and Toddler Development, Third Edition (BSID-IV)

Z-scores and percentiles for weight-for-age (WFA) and stature-for-age (SFA) in subjects <18 years of age on the date of informed consent (Part C only)

### Pharmacokinetics:

Plasma will be analyzed by liquid chromatography with tandem mass spectrometry (LC-MS/MS) with a lower limit of quantitation of 0.100 μg/mL. Samples collected for analyses of Compound 5 (plasma/serum/whole blood)] concentration may also be used to evaluate safety or efficacy aspects related to concerns arising during or after the study.

Blood samples for measuring Compound 5 concentrations will be collected from each subject at specified time points.

Plasma PK concentration values will be listed and summarized at each scheduled time point by day/dose level. The data will be summarized by descriptive statistics. Concentration data from all parts of the study may be combined for a population PK (popPK) analysis.

### Statistical Considerations:

### Rationale for Sample Size:

The study plans to enroll a minimum of 12 evaluable subjects. All subjects will initially be evaluated during a 4-week run-in period followed by within-subject dose escalation of 3 doses (1, 3, and 10 mg/kg QD) to check for improvement in disease-related biomarkers. The results of Part A will be used to determine the dose level to be used in Parts B and C. At the end of Part A, subjects will continue into a 4-week washout period to determine the time for disease-related biomarkers to return to baseline levels. If this evaluation shows that the duration of the washout period needs to be altered, the crossover washout in Part B will be adjusted.

Part B will enroll a minimum of 12 evaluable subjects and is a 6-month randomized, double-blind, placebo-controlled, 2-period crossover study with a 4-week washout in between the 12-week treatment periods.

Randomization will be stratified by disease type to ensure a balance of sequences within each disease type. Any subject who discontinues during Part A or prior to having a post-dose efficacy assessment in the second period of Part B will be replaced with a subject of the same disease type, assigned to the same treatment sequence, to maintain balance in the design.

A DMC will be convened to assess the safety of subjects in Parts A, B, and C, and it may recommend changes to the enrollment of subjects in Part A, B, and/or C.

In Part A, a sample size of 12 subjects per dose level (6 PA and 6 MMA) is adequate to evaluate the initial safety, PK, and PD of Compound 5 based on the expected improvements in disease-related biomarkers. The ability of Compound 5 to reach steady-state concentrations within a few days (Perrine, 2011), coupled with the expected rapid, large, and reversible responses of disease-related biomarkers to Compound 5 in PA and MMA subjects, make within-subject dose escalation a more feasible and efficient study design than dosing different subjects at each dose level. The study will target approximately 3 subjects per age group (≥2-11 and ≥12 years of age) for each disease (PA and MMA) for a total of 12 subjects to ensure a representative distribution of subjects for PK, PD, and safety assessments.

In Part B, with a combined sample size of 12 subjects, there will be 97% power to detect an absolute difference of 50% in percent change from baseline in plasma MCA levels for Compound 5 compared with placebo using a paired t-test at a two-sided α=0.05 and assuming a standard deviation of paired differences of 40% (this standard deviation assumes heterogeneity across the 2 diseases).

### Statistical Analysis Plan (SAP):

All subjects who receive at least 1 dose of study drug and have at least 1 efficacy assessment post-baseline will be included in the Full Analysis Set (FAS), which will be defined separately for each study part.

Efficacy analyses will be based on the FAS, except for the primary efficacy analysis in Part B, which will be based on the Crossover Evaluable Analysis Set. The Crossover Evaluable Analysis Set will include all FAS subjects who have at least 1 post-baseline efficacy assessment in each of the 2 treatment periods in Part B. In case of a dropout and replacement, only the replacement subject will be included in this analysis set. All subjects who take at least 1 dose of study treatment will be included in the Safety Analysis Set, which will be identified separately for Parts A, B, and C. Baseline and safety analyses will be based on the Safety Analysis Set. PK analyses will be performed on the PK analysis set, defined as all subjects who receive any amount of Compound 5 and have enough samples collected to permit analyses. The PK analysis set will also be identified separately for Parts A, B, and C.

Continuous laboratory data will be examined for trends using descriptive statistics of actual values and changes from baseline over time. Shift tables from baseline to each post-baseline time-point will be presented. Vital signs will be summarized using descriptive statistics of actual values and changes from baseline over time. The incidence of clinically notable vital signs will be summarized. Physical examination findings will be presented in subject listings.

AEs will be mapped to preferred term and system organ class using the Medical Dictionary for Regulatory Activities (MedDRA). AEs that begin after the first administration of investigational products or existing AEs that worsen after the first dose of study medication are considered TEAEs. For Part A, each TEAE will be assigned to a given dose based on its start date. A TEAE will be assigned to a given dose level if it starts or worsens in severity on or after the first administration of study medication at that dose level but before the first administration of study medication at the next dose level. For Part B, a TEAE will be assigned to either Compound 5 or placebo if it starts or worsens in severity on or after the first administration of the study treatment but before the first administration of the subsequent treatment. Part C TEAEs will be summarized by disease and overall.

The number and percentage of subjects reporting TEAEs will be summarized by MedDRA system organ class and preferred term, by severity, and by relationship to study treatment. Drug-related AEs will be considered those to be at least possibly related to investigational product based on the Investigator's assessment. The number and percentage of subjects with serious AEs (SAEs), and the number and percentage of subjects with AEs leading to treatment discontinuation will also be summarized by MedDRA system organ class and preferred term.

The primary efficacy endpoint in Part A is the within-subject percent change from baseline in fasting (for minimum of 3 hours) plasma MCA levels measured at weeks 2 and 4 during the following 4-week periods: baseline through week 4 (run-in), weeks 5 through 8, weeks 9 through 12, and weeks 13 through 16 (corresponding to 4-week treatment periods ending with dose levels of 1, 3, and 10 mg/kg Compound 5A QD, respectively; 1.0 mg of Compound 5A equals 0.84 mg of Compound 5). Percent change from baseline will be calculated as [100*(post-baseline value −baseline)/baseline]. Descriptive statistics for observed, change from baseline, and percent change from baseline in MCA levels will be presented by dose for each disease (PA or MMA) and overall. Pairwise comparisons between dose levels (0, 1, 3, and 10 mg/kg) using the corresponding 95% CIs for means of within subject dose differences will also be presented by disease and overall. Similar within-subject analyses will be performed for the washout period primary endpoint comparing the on-treatment percent change from baseline at week 16 with the corresponding off-treatment averages post-washout.

The primary endpoint analyses for Part A will be based on a mixed-effect model repeated measures (MMRM), which will be fitted to all measured percent change from baseline in fasting (minimum of 3 hour) plasma MCA levels and will include fixed effects for dose level, disease, weeks-on-dose-level, dose level-by-disease interaction, dose level by weeks-on dose level interaction as well as a random effect for subject. The LS mean estimates based on the MMRM model will be used for pairwise comparisons of dose levels overall and for each disease. Pairwise comparison 95% CIs for the differences in LS means will be displayed. A dose-response analysis on the MCA levels in Part A will be conducted. Mean MCA levels (on vertical axis) will be presented graphically by dose (on horizontal axis). Assessments of linear and quadratic dose-response trends across doses will be performed using the MMRM model with appropriate orthogonal polynomial contrasts. P-values for each orthogonal polynomial contrast will be presented. It should be noted that the MMRM model will be used to implicitly impute for missing data under the missing at random (MAR) assumption.

The primary efficacy endpoint in Part B is within-subject percent change from baseline in fasting (for minimum of 3 hours) plasma MCA levels, measured every 4 weeks during each treatment period. Baseline will be defined as the latest plasma MCA measurement prior to the first dose of study treatment in each treatment period in Part B. Descriptive statistics for observed, change, and percent change from baseline will be presented by treatment for each disease (PA or MMA) and overall. Comparisons between treatments (Compound 5 and placebo) using the corresponding 95% CIs for means of within subject differences measured at the end of each period will also be presented by disease and overall.

The Part B primary endpoint analyses will be based on an MMRM, which will be fitted to all measured percent change from baseline in fasting (minimum of 3 hours) plasma MCA levels and will include fixed effects for disease, treatment (Compound 5 or placebo), nominal week on treatment (4, 8, or 12), treatment-by-week interaction, sequence as well as a random effect for subject nested within sequence. The LS mean estimates based on the MMRM model will be used for comparisons of treatment overall and for each disease, by nominal week and over the entire treatment period. Treatment comparison p-values and the corresponding 95% CIs for the differences in LS means will be displayed. In addition, the fixed effect of sequence will be tested to determine whether there is a carryover between treatment periods. For exploratory estimates within disease, the model may be run for each disease separately.

Frequency of episodes and frequency of days requiring hospitalization or an ER visit for metabolic decompensations are secondary efficacy endpoints for Part B and will be analyzed similarly to the MCA levels. Detailed descriptions of all statistical analyses will be provided in the SAP.

### Example 8

Phase 2 Study of Compound 5 in Subjects with Propionic or Methylmalonic Acidemia Continued

The doses used in the clinical trial described in Example 6 are expanded to include doses of about 3 mg/kg, about 9 mg/kg, and about 15 mg/kg of Compound 5A, administered twice daily (corresponding to total daily doses of 6 mg/kg/day, 18 mg/kg/day and 30 mg/kg/day, respectively). The doses correspond to about 2.5 mg/kg, about 7.6 mg/kg, and about 12.6 mg/kg of Compound 5, respectively. Primary and secondary outcomes, and PK were measured according to Example 6.

### Protocol Part A: Within-Subject Dose Escalation and Open-Label Extension

Part A is the dose escalation phase to assess the initial safety and to confirm the pharmacologic activity of Compound 5 in subjects with PA and MMA. Enrollment took place within 28 days (or longer if approved by the medical monitor) after screening for eligibility. All subjects were initially evaluated during a 4-week run-in period followed by within-subject dose escalation of 3 dose levels. The 4-week run-in period could be extended if the subject had an illness that required hospitalization or a change in dietary therapy and needed time (up to 2 weeks) to recover to his/her usual state of health. Subjects received 3, 9, and 15 mg/kg BID in sequential 4-week dose intervals to evaluate improvements in disease-related biomarkers. An internal SRC was responsible for an ongoing review of all available safety, PK, and efficacy data during dose escalations in Part A of the study. Additionally, an independent DMC reviewed all available safety, PK, and efficacy data quarterly and prior to the start of Part B of the study.

Dose limiting toxicity (DLT) is defined as a Grade 3 or higher AE (based on the Common Terminology Criteria for Adverse Events version 5.0 [CTCAE v5.0]) considered related to Compound 5. If a subject experienced a DLT, further dosing of the subject was stopped until the AE resolves to Grade 1 or to the baseline level. In addition, if the subject experienced a Grade 1 or 2 AE considered related to Compound 5, further dosing of the drug could be stopped at the discretion of the Investigator after consultation with the Sponsor until the AE resolved to Grade 1 or baseline level. After discussion between the SRC and the Investigator, the subject could resume dosing at the same dose, the next lower, or an intermediate dose level, and barring any safety concerns, re-escalated as tolerated. If 2 subjects experienced the same Grade 3 or higher AE or any single subject experienced a Grade 4 or higher AE, either of which, in the opinion of the Investigator, were considered related to HST5040, enrollment and dosing of additional subjects was stopped, and an ad hoc DMC meeting convened to provide recommendations for the Sponsor's consideration regarding continuation, modification, or discontinuation of dosing. Subjects who do not complete dose escalation were replaced.

At the end of Part A, subjects will continue to be monitored in the study for an additional 4 weeks without Compound 5 drug treatment, according to the schedule of assessments. The purpose of this washout period is to determine the time for disease-related biomarkers to return to baseline levels, and thus, the duration of the carry-over effect of pharmacologic activity on disease-related biomarkers. If this evaluation shows that the duration of the washout period needs to be altered, the crossover washout in Part B will be adjusted (see below). This information will be informative for the design of future studies.

After individual subjects complete the washout period, they will receive Compound 5 at their highest tolerated dose in an open-label extension of Part A. Monthly PK/PD and safety assessments will be conducted according to the schedule of assessments. This extension of treatment will continue until all subjects have completed the dose escalation, the data have been analyzed, and the optimal dose and regimen of Copmound5 has been determined for Parts B and C.

At the end of the Part A extension period, subjects will undergo a minimum 4-week washout period prior to the start of Part B described in the above example.

The 4-week washout periods at the end of Part A and the end of Part A Extension may be extended if the subject has an illness that requires hospitalization or a change in dietary therapy and needs time (up to 2 weeks) to recover to his/her usual state of health.

### Example 9

### A Study of Preliminary Exposure Data and Projected Exposures Based on Simulations

Preliminary pharmacokinetics (PK) data from the first two subjects of Study described in Examples 7-8 was evaluated at 1 mg/kg, 3 mg/kg and 10 mg/kg of Compound 5 dosed once daily (QD). Results from the preliminary analysis are shown in Table 6. Exposure appears to be slightly lower than what was projected from the previously established PK model (see Projected Values in Table 6); however, the 3 mg/kg and 10 mg/kg dose still provided exposures above the estimated in vitro EC90 (2.32 μg/ml).

TABLE 6
Summary of Exposures Observed from Study and Projected Exposures Based on Simulations

| Dose | | Cₘₐₓ: | Cₘₐₓ: | AUC_{0-24 h} | AUC_{0-24 h}: | C24 | C24: |
|---|---|---|---|---|---|---|---|
| (QD) | Subject | (ug/mL) | C_{max(projected)}* | (ug*h/mL) | AUC_{projected} | (ug/mL) | C24_{projected}* |
| 1 mg/kg | 010-1001 | 2.62 | — | 30.8 | — | — | — |
| SD | 010-1002 | 1.74 | — | 22.8 | — | — | — |
| 1 mg/kg | 010-1001 | 4.41 | 0.706 | 56.5 | 0.710 | 1.17 | 1.27 |
| SS | 010-1002 | 2.93 | 0.469 | 36.5 | 0.459 | 0.765 | 0.829 |
| | Projected* | 6.25 | — | 79.6 | — | 0.923 | — |
| | Value | | | | | | |
| 3 mg/kg | 010-1001 | 15.9 | 0.850 | 189 | 0.791 | 3.34 | 1.21 |
| SS | 010-1002 | 11.4 | 0.610 | 115 | 0.481 | 1.01 | 0.365 |
| | Projected | 18.7 | — | 239 | — | 2.77 | — |
| | Value* | | | | | | |
| 10 mg/kg | 010-1001 | 50.7 | 0.811 | 557 | 0.700 | 9.09 | 0.985 |
| SS | 010-1002 | 50.6 | 0.810 | 678 | 0.852 | 13.1 | 1.42 |
| | Projected | 62.5 | — | 796 | — | 9.23 | — |
| | Value* | | | | | | |

SD = single dose;
SS = steady-state;
Cₘₐₓ = maximum concentration;
AUC_{0-24 h} = area under the concentration-time curve from 0 to 24 hours;
C₂₄ = concentration at 24 hours post-dose

To increase exposure levels, doses of 3 mg/kg to 15 mg/kg were administered twice daily. Based on the previous PK model and assuming dose proportionality, projected steady-state exposures for 3, 9 and 15 mg dosed twice daily are described in Table 7.

TABLE 7
Summary of Projected Steady-State Exposures
for 3, 9 and 15 mg BID Dosing

| Dose | Cₘₐₓ | C₁₂ | AUC_{0-12 h} |
|---|---|---|---|
| (BID) | (ug/mL) | (ug/mL) | (ug*h/mL) |
| 3 mg/kg | 26.3 (28) | 11.6 (77) | 238 (23) |
| 9 mg/kg | 78.8 (28) | 34.7 (77) | 715 (23) |
| 15 mg/kg | 131 (28) | 57.9 (77) | 1190 (23) |

Data are presented as geometric mean (CV %) of 100 simulated subjects, adjusted assuming dose proportionality.
Cₘₐₓ = maximum concentration;
C₁₂ = concentration at 12 h post-dose;
AUC_{0-24 h} = area under the concentration-time curve from 0 to 12 h

### INCORPORATION BY REFERENCE

It should also be understood that all patents, publications, journal articles, technical documents, and the like, referred to in this application, are hereby incorporated by reference in their entirety and for all purposes.

The various embodiments described above and throughout the specification can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary, to employ concepts of the various patents, application and publications to provide yet further embodiments

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1-51. (canceled)

52. A method of treating an organic acidemia in a subject in need thereof, comprising:
administering about 0.5 mg/kg to about 30 mg/kg of 2,2-dimethylbutyric acid, or an equivalent amount of a pharmaceutically acceptable salt or ester thereof, to the subject,
thereby treating the organic acidemia.

53. A method of treating propionic acidemia or methylmalonic acidemia in a subject in need thereof, comprising:
orally administering about 1 mg/kg to about 30 mg/kg of sodium 2,2-dimethylbutanoate, or an equivalent amount of 2,2-dimethylbutyric acid or a different pharmaceutically acceptable salt or ester thereof, to the subject,
thereby treating propionic acidemia or methylmalonic acidemia.

54. The method of claim 53, wherein the organic acidemia is propionic acidemia.

55. The method of claim 53, wherein the organic acidemia is methylmalonic acidemia.

56. The method of claim 53, wherein about 1 mg/kg, about 3 mg/kg, about 6 mg/kg, 9 mg/kg, about 10 mg/kg, about 15 mg/kg, about 18 mg/kg, or about 30 mg/kg of sodium 2,2-dimethylbutyrate is administered to the subject per day.

57. The method of claim 56, wherein sodium 2,2-dimethylbutyrate is formulated as an oral solution.

58. The method of claim 57, wherein the oral solution comprises from about 5 mg/ml to about 75 mg/mL of sodium 2,2-dimethylbutyrate.

59. The method of claim 57, wherein the oral solution comprises from about 10 mg/mL to about 70 mg/mL of sodium 2,2-dimethylbutyrate.

60. The method of claim 53, wherein the treating comprises reducing propionic acid, 3-hydroxypropionic acid, methylcitrate, glycine, propionylcarnitine, or combinations thereof in the subject compared to baseline or a control.

61. The method of claim 53, wherein the treating comprises reducing 2-ketoisocaproate, isovaleryl-CoA, 3-methylcrotonyl-CoA, 3-methylglutaconyl-CoA, 3-OH-3-methylglutaryl-CoA, 2-keto-3-methylvalerate, 2-methylbutyryl-CoA, tiglyl-CoA, 2-methyl-3-OH-butyryl-CoA, 2-methyl-acetoacetyl-CoA, 2-ketoisovalerate, isobutyryl-CoA, methylacrylyl-CoA, 3-OH-isobutyryl-CoA, 3-OH-isobutyrate, methylmalonic semialdehyde, propionyl-CoA, or methylmalonyl-CoA, or combinations thereof, in the subject compared to baseline or a control.

62. The method of claim 60, wherein the at least one metabolite is reduced by an amount ranging from at least about 10% to about 100%.

63. The method of claim 61, wherein the at least one metabolite is reduced by an amount ranging from at least about 10% to about 100%.

64. The method of claim 53, wherein about 3 mg/kg of sodium 2,2-dimethylbutanoate is administered twice a day.

65. The method of claim 53, wherein about 9 mg/kg of sodium 2,2-dimethylbutanoate is administered twice a day.

66. The method of claim 53, wherein about 15 mg/kg of sodium 2,2-dimethylbutanoate is administered twice a day.

67. The method of claim 53, wherein the subject is 2 to 11 years old.

68. The method of claim 53, wherein the subject is 12 years old or older.

69. The method of claim 53, wherein the subject is 2 years old or younger.

70. The method of claim 53, wherein the total daily dose of sodium 2,2-dimethylbutanoate administered is about 6 mg/kg.

71. The method of claim 53, wherein the total daily dose of sodium 2,2-dimethylbutanoate administered is about 18 mg/kg.

72. The method of claim 53, wherein the total daily dose of sodium 2,2-dimethylbutanoate administered is about 30 mg/kg.

73. The method of claim 53, wherein the treating comprises reducing MCA levels, plasma MCA: citric acid ratio, plasma propionyl-carnitine to acetyl carnitine ratio, 3-OH propionate levels, propionyl-carnitine levels, ammonia (NH₃) levels, anion gap, carnitine levels, ketone levels, lactic acid levels, ketone urine levels, organic acid levels, acylglycines levels, QT corrected (QTc) intervals, a left ventricular ejection fraction, plasma and urinary metabolites, plasma fibroblast growth factor 21 (FGF-21) levels, or combinations thereof, in the subject compared to baseline or a control.

74. The method of claim 53, wherein the treating comprises reducing a frequency of metabolic decompensation events requiring an emergency room visit or hospitalization; a frequency of days in the hospital required for treatment/resolution of metabolic decompensations; and/or frequency of episodes and/or days requiring use of a home emergency treatment protocol for metabolic decompensations, in the subject compared to baseline or a control.

75. The method of claim 53, wherein the subject exhibits an increase in plasma sodium 2,2-dimethylbutyrate levels after administration.

76. The method of claim 53, wherein the treating comprises improving health-related quality of life (HrQoL) in the subject.

77. The method of claim 76, wherein the treating comprises decreasing a MetabQOL 1.0 score of the subject by 1, 2, 3, or 4.

78. The method of claim 76, wherein the treating decreases a PedsQL score of the subject by 1, 2, 3, or 4.

79. The method of claim 76, wherein the treating decreases Clinician-Reported Global Assessments of Severity and Change score of the subject by 1, 2, 3, 4, 5, or 6.
